(19) United States
(12) Patent Application Publication
(10) Pub. No.: US 2025/0262365 A1
(43) Pub. Date: Aug. 21, 2025

(54) PULMONARY EMBOLISM EXTRACTION DEVICE

(71) Applicant: TRIFORTIS LLC, Tucson, AZ (US)

(72) Inventors: Dexter De Leon, Tucson, AZ (US); Phat Le Tran, Tucson, AZ (US); David Moore, Tucson, AZ (US); Cody Mitts, Tucson, AZ (US)

(21) Appl. No.: 19/201,757

(22) Filed: May 7, 2025

Related U.S. Application Data
(63) Continuation-in-part of application No. 17/746,812, filed on May 17, 2022.
(60) Provisional application No. 63/189,331, filed on May 17, 2021.

(51) Int. Cl. A61M 1/34; A61M 1/36; A61M 25/06; A61M 29/00; A61M 39/02; A61M 60/113; A61M 60/258; A61M 60/279; A61M 60/37

(52) U.S. Cl. CPC A61M 1/34; A61M 1/3626; A61M 1/3635; A61M 1/3659; A61M 29/00; A61M 39/0247; A61M 60/113; A61M 60/258; A61M 60/279; A61M 60/37; A61M 2025/0681; A61M 2039/0258; A61M 2039/0273; A61M 2039/0276; A61M 2039/0279; A61M 2202/0427; A61M 2205/3334; A61M 2205/502

(57) ABSTRACT

A device for extracting arterial and pulmonary embolisms. Blood containing unwanted material is suctioned out of a patient, is filtered in the reservoir, and is returned to the patient. The device includes a suction catheter and a return catheter attached to a filter reservoir. The device may also include a daughter catheter to reach smaller spaces and a fishing catheter to catch and reel the unwanted material toward the suction catheter. The reservoir is a two-stage filter that filters out any unwanted material from the blood and de-airs the blood prior to returning the blood back to the patient. The suction system can be manual or controlled by a console integrated with computer readable instructions and algorithms to safely return the filtered and de-aired blood.

### CROSS-REFERENCES TO RELATED APPLICATIONS

This application is a continuation-in-part and claims benefit of U.S. Non-Provisional application Ser. No. 17/746,812 filed May 17, 2022, which is a non-provisional and claims benefit of U.S. Provisional Application No. 63/189,331 filed May 17, 2021, the specification of which is incorporated herein in its entirety by reference.

### BACKGROUND OF THE INVENTION

The present invention relates to the removal of foreign material, in situ or embolic thrombus in the vascular tree like intracardiac thrombus and pulmonary embolism using the devices and methods described herein.

Pulmonary embolism is a blockage in the pulmonary arteries of the lungs. In most cases, pulmonary embolism is caused by blood clots that travel to the lungs from deep veins in the legs or, rarely, from veins in other parts of the body (deep vein thrombosis). Since the clots block blood flow to the lungs, pulmonary embolism can be life-threatening. However, prompt treatment greatly reduces the risk of death.

Current devices used for embolism extraction have outdated designs; where they aspirate blood and clots then subsequently discard of themOne of these mechanical suction devices comprises a large catheter with a 60 ml syringe at the back to apply negative pressure for aspiration. The instruction for use includes: A) Floating a pigtail to the pulmonary artery; B) Delivering suction to aspirate the blood and the thrombus; and C) Discarding the aspirated blood. There are several issues with the use of this device. It is very time consuming and blood loss during the procedure is very significant. The pigtail is not designed to be floated to the pulmonary artery due to its stiffness and shape. Most operators use a wire and wire the right ventricle to the pulmonary artery, then pass the pigtail through the wire. This procedure is unsafe because the wire can pass through the tricuspid apparatus which can then be damaged as the catheter passes through the apparatus. In another thrombectomy device, the company uses a 30 ml syringe aspiration system and discards the blood into a waste bag. The use of a 60 ml or 30 ml syringe to aspirate blood and clots and subsequently discarded can be detrimental due to blood loss. In some cases, up to about 1.5 L of blood can be aspirated, and after the procedure, the patient is anemic and hypotensive. Furthermore, the blood loss may then increase mortality. Multiple studies in cardiology have linked blood loss to poor outcomes.

In another mechanical suction system, a catheter is intended for use in procedures requiring the extracorporeal circulatory bypass support. Therefore, the aspirated blood may be re-infused simultaneously back to the patient. The system can minimize blood loss, but it still has many limitations that can generate adverse events. The system cannot handle air and the catheter cannot reach the pulmonary trunk. It is mainly used to aspirate materials in the right heart chambers. The system also requires a specialist to operate and aspirated materials cannot be seen readily for treatment assessment. In some cases, the filter can be clotted by the materials causing an exchange that can be detrimental to the patient. Additionally, crystalloid, therapeutics or blood products cannot be added with the device.

### BRIEF SUMMARY OF THE INVENTION

It is an objective of the present invention to provide devices and methods that allow for the aspiration of foreign bodies, clots, or infection material from a blood vessel and cardiac chambers, as specified in the independent claims. Embodiments of the invention are given in the dependent claims. Embodiments of the present invention can be freely combined with each other if they are not mutually exclusive.

The present invention features a device for aspirating blood from a patient. Blood is aspirated to remove a foreign body, a clot, or other infectious material from blood vessels and cardiac chambers. The aspirated blood is then filtered, de-aired, and transfused back to the patient safely via a console configured to execute computer-readable instructions. This process makes clot removal more efficient with no impact on blood loss. To prevent hemodynamic compromise, the computer-readable instructions may comprise reading the central venous pressure or preload volume of the heart. The volume of blood that is suctioned out is the same volume of blood being delivered back to the patient to maintain preload pressure. The device may have a graded delivery of negative suction. As the clot is engaged, the pressure is increased. If the suctioned blood flow is greater than 1 liter per minute or the preload pressure is compromised, then negative suction pressure is decreased by the console. The computer-readable instructions may further comprise increasing the return of the blood or volume to maintain homeostasis. The device comprises a reservoir that acts as a large filter and air trap. Air handling is an important part of the design as air should not be introduced into the patient's circulation. Furthermore, the device can be integrated to a cardiopulmonary bypass machine for hemodynamic support, or the device can also be used as a rapid transfuser of blood, therapeutics and crystalloid products.

One of the unique and inventive technical features of the present invention is the use of a long flexible catheter, a tapered flexible daughter catheter, and a fishing catheter that can reach a smaller area of the pulmonary artery. Current devices can place a catheter in the heart and are typically too rigid to place in the pulmonary artery. In the event that the long, flexible catheter cannot reach a smaller area of the pulmonary artery, a tapered flexible daughter catheter in the present invention can be added as an extension of the first catheter to extract unwanted materials in the smaller vessels. Alternatively, a fishing catheter that can be deployed through the screw-in lock cap to extract the unwanted material toward the first catheter. In yet another embodiment, the mesh at the end of the fishing catheter is configured to be expanded when needed and in multiple configurations of a satellite-like, an umbrella-like or a helical-like mesh structure as desired by the end user.

Another unique and inventive technical feature of the present invention is the automated application of negative pressure to aspirate blood/clot or any material from the patient. The system has a computer readable instruction console which regulates and automates a graded delivery of negative pressure to provide ample negative suction while preventing hemodynamic compromise. As the blood is being aspirated from the patient, the blood is filtered in the reservoir of the device to remove/separate foreign bodies, clots, or other infectious material and also to remove bubbles and air, before the blood is transfused back to the patient. The conical shape of the funnel and the half-wall spill plate disc in the reservoir ensures that there is laminar flow of the blood as it is filtered, thus minimizing damage to the blood and eliminating the production of micro bubbles. The console, which can read and be incorporated with the patient's vital parameters; coupled with multiple sensors and algorithms, can semi autonomously reinfuse filtered, de-aired blood safely.

Without wishing to limit the invention to any theory or mechanism, it is believed that the technical features of the present invention advantageously ensure patient safety by preventing significant blood loss from the patient. The amount of blood that is aspirated from the patient is the same amount that is transfused back to the patient. Furthermore, the device described herein also eliminates air bubbles when filtering blood and can act as a rapid infuser to transfuse additional volume like crystalloids, salines or blood. None of the presently known prior references or work has the unique inventive technical features of the present invention.

Another one of the unique and inventive technical features of the present invention is the implementation of a filter system having a funnel structure, coupled fluidly to a specialized spill plate. Without wishing to limit the invention to any theory or mechanism, it is believed that the technical features of the present invention advantageously allows for the laminar flow of the blood as it is filtered, thus minimizing damage to the blood and eliminating the production of microbubbles. The laminar flow allows for constant and automatic control of the flow rate of blood as it travels through the filter system. Furthermore, the laminar flow of blood causes air to rise into the upper chambers of the filter system without mixing with the fluid itself, thus minimizing microbubbles and the chance for air embolisms upon reinfusion of the blood into the body. Furthermore, the slidable spill plate supports the funnel structure and promotes further laminar blood flow to the bottom of the filter system, thus further preventing the formation of microbubbles in the blood flow. None of the presently known prior references or work has the unique inventive technical features of the present invention.

Another one of the unique and inventive technical features of the present invention is the implementation of a console operatively coupled to the filter system and the sensors, configured to control pressure or flow rates of the suction flow and the return flow based on the fluid level of the blood in the filter system such that the suction flow, the return flow, or a combination thereof are stopped by the console when bubbles are detected, when the fluid level is below a threshold, or a combination thereof. Without wishing to limit the invention to any theory or mechanism, it is believed that the technical features of the present invention advantageously allows for the prevention of microbubbles from entering the return flow. This further minimizes the chance for air embolisms upon reinfusion of the blood into the body. None of the presently known prior references or work has the unique inventive technical features of the present invention.

Another one of the unique and inventive technical features of the present invention is the implementation of a console operatively coupled to the filter system and the plurality of sensors, comprising a processor configured to execute computer-readable instructions and a memory component operatively coupled to the processor, comprising computer-readable instructions. Without wishing to limit the invention to any theory or mechanism, it is believed that the technical features of the present invention advantageously allows the presently claimed invention to effectively determine the status of the blood in the filter system and ensure that the introduction of air into the filtered blood is minimized. The console is configured to detect the presence of bubbles, the balance of the suction flow and the return flow, and the level of fluid in the filter reservoir. Depending on any one of these parameters, the console is configured to either allow flow to continue through the filter or stop the flow until the parameters are within acceptable limits. Furthermore, the console is configured to prevent low fluid levels, which allows for the prevention of microbubbles from entering the return flow. This further minimizes the chance for air embolisms upon reinfusion of the blood into the body. The console also intake patient parameters such that if the patient needs more volume resuscitation, the console will transfuse more volume by adjusting the return flow rate and suction flow rate. None of the presently known prior references or work has the unique inventive technical features of the present invention.

In the absence of the console, a manual syringe pump can be implemented to extract unwanted materials and to return the desired blood. The unique and inventive technical features of the present invention is a large lockable syringe quick-connect embedded in a T-fashion between two duckbill one-way valves. The direction of the one-way valve dictates the flow. When the syringe is pulled and locked, it will generate a negative suction to drain blood/clots/unwanted materials through the one-way valve. When it is pushed, the blood/unwanted materials will be pushed through the other one-way valve. The unwanted materials will be filtered and only the de-aired or desired blood is to be returned to the patient.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a schematic diagram of an integrated embolism extraction system consisting of catheters connecting to the reservoir and operating by the computer readable instruction console.

FIGS. 2A-2C show CAD drawings of the integrated filter reservoir of the present invention. FIGS. 2A-2B show external views of the reservoir showing drainage connection ports at the top of the reservoir, vacuum port at the middle chamber, and returning port at the bottom. FIG. 2C shows the internal set-up of the reservoir; demonstrating the top chamber with an upper filtration system in a pyramid like structure, where clots are filtered and blood slide down a transparent solid funnel-like structure to a 2nd defoaming system where blood is further de-air to eliminate microembolism through a half-walled sliding spill plate that force blood to undergo laminar flow toward a slide that further de-air the blood before being returning to the patient.

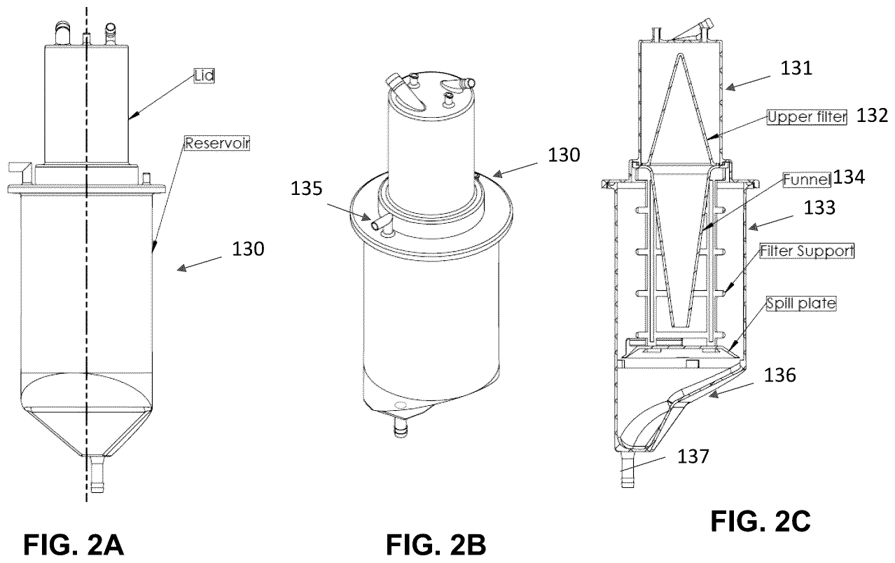

FIGS. 3A-3B show CAD drawings of the return catheter illustrating a side port that can be connected to return blood (FIG. 3A) and an end with seal membrane (FIG. 3B) that can be used to wire the cannula with minimal bleeding. The cannula is specifically designed long enough to return blood to the right heart so that hemodynamic instability can be minimized.

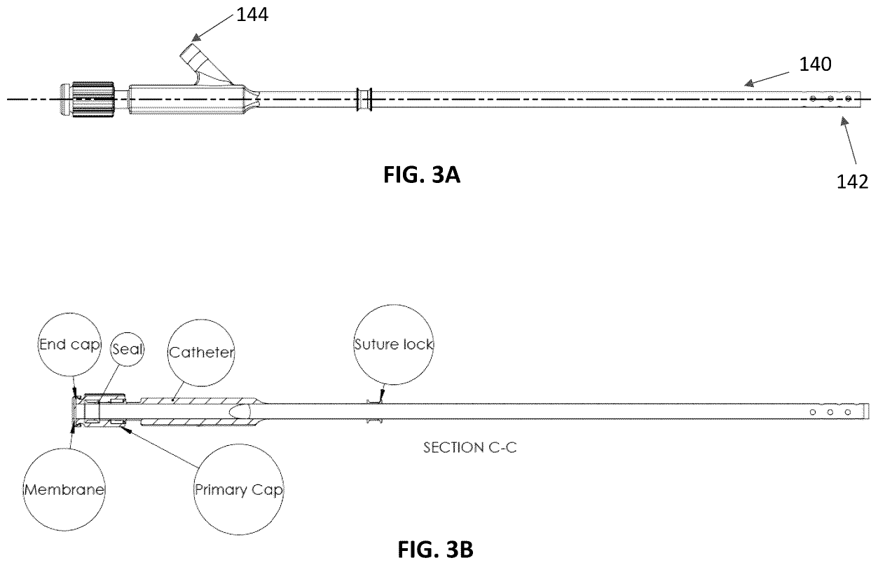

FIGS. 4A-4C show CAD drawings of the suction catheter that is wire enforced (FIG. 4B) to prevent collapse from high negative suction pressure The drainage of the blood is also on the side of the catheter in a 45 degree slanted angle (FIG. 4A). The proximal end of the cannula also has an end cap membrane to prevent bleeding and provide an access for dilators, daughters, fishing catheters, and wires. The distal end is flexible and bendable to reach the heart and the pulmonary artery (FIG. 4C).

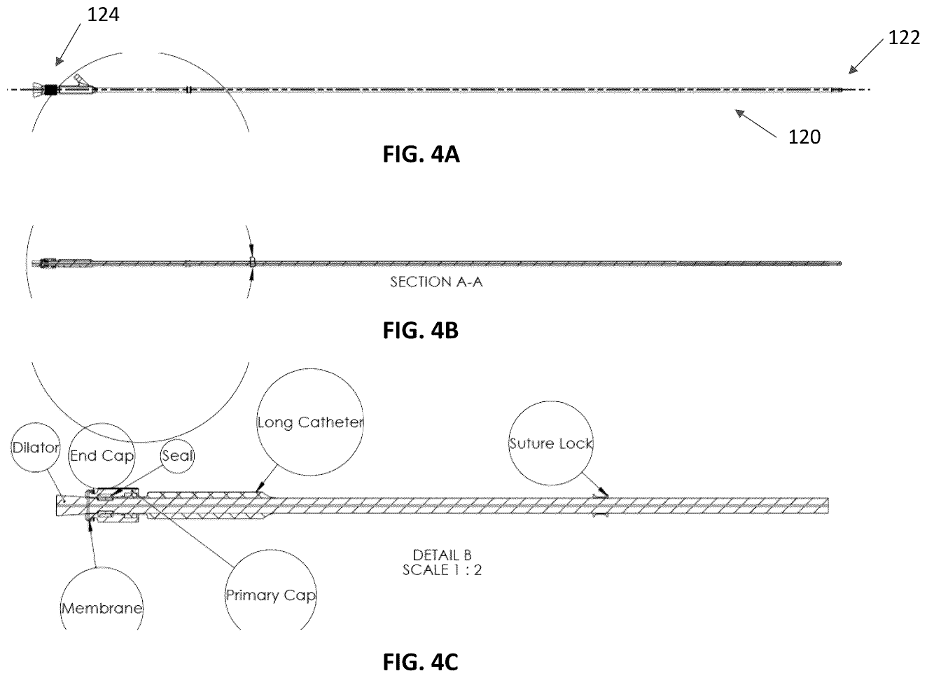

FIG. 5A shows a CAD drawing of a console system configured to execute computer readable instructions and algorithms of the present invention to properly remove foreign materials and safely return filtered, de-aired blood.

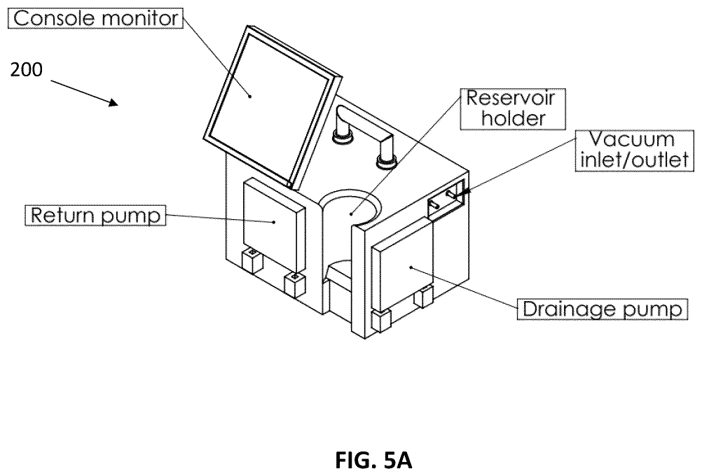

FIG. 5B shows a CAD drawing of a top view of a pedal that may be connected to the console to control the system. The left pedal is for drainage or suction. The middle pedal is for turning the console on/off, and the right pedal is for transfusion or returning the filtered, de-aired blood.

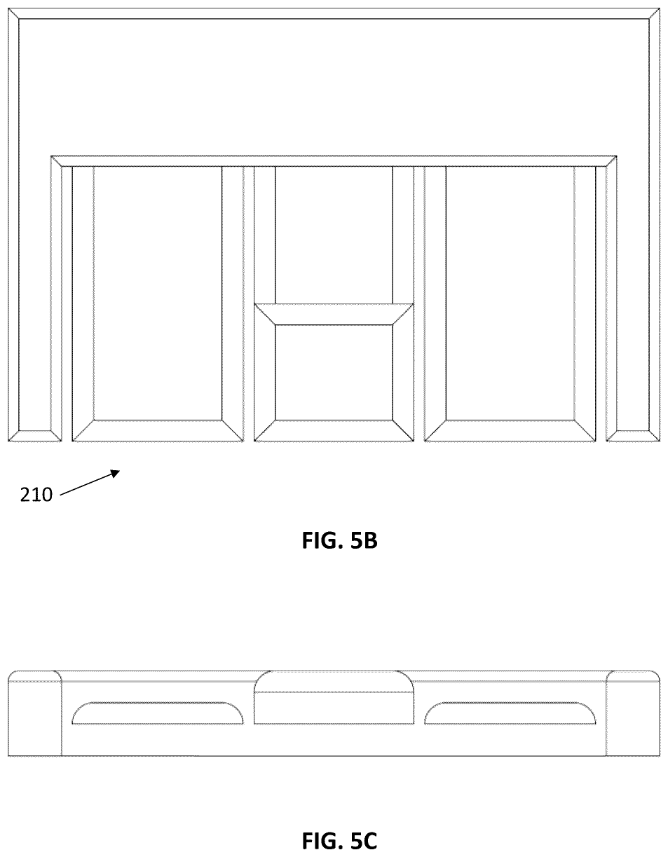

FIG. 5C shows a CAD drawing of a side view of the pedal.

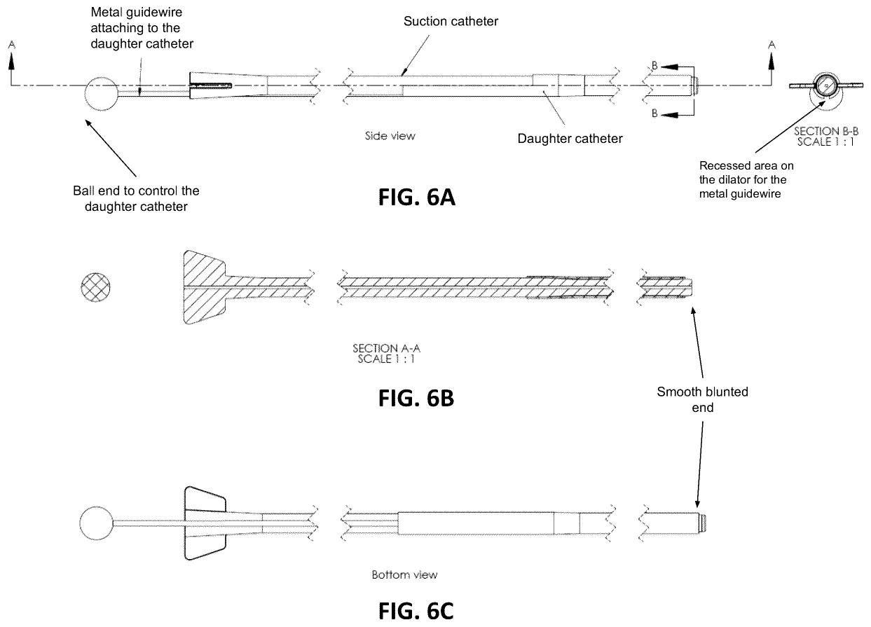

FIGS. 6A-6C show CAD drawings of the tapered daughter catheter embedded with recessed dilators that act like the extension of the suction catheter to reach a smaller area of the lung that the suction catheter cannot get to.

FIGS. 7A-7C show complete CAD drawings of a fishing catheter with a nitinol wire at the distal end that can be deployed into a satellite-like or umbrella-like or helical-like mesh structure. FIG. 7A shows a diagram of the fishing catheter of the present invention with 2 side ports that can be used to de-air the catheter but can also hook up to a pressure sensor gauge to monitor the pressure at the tip of the catheter. The outer sideport like a pig tail is used to de air the long sheath. The sheath will encapsulate the nitinol mesh to prevent scratching/damaging the vessels during insertion. Once in the proper place to retrieve clots or foreign materials, the sheath is pulled back to deploy the nitinol mesh.

FIG. 7B shows a cross sectional image of the retriever catheter, depicting the inner section with the presence of the cap and seal to prevent bleeding. FIG. 7C shows the inner section of the retriever catheter comprising a rod that extends to the distal end of the mesh wire; whereas the other side of the mesh wire is connected to the end of the outside catheter.

FIGS. 8A-8D show flowchart diagrams of subroutine algorithms that are being executed sequentially in a loop by the console to stop or control flow to or from the reservoir. FIG. 8A shows a flowchart of an algorithm for a system check for bubbles, flow sensors, and fluid level engagement prior to initiation. FIG. 8B shows a flowchart of an algorithm for a check for bubbles. This subroutine comprises waiting for a signal from a sensor that indicates an air bubble has been detected. If not, the operation continues as normal. If a bubble is detected all operation stops. FIG. 8C shows a flowchart of an algorithm for a check for fluid levels. FIG. 8D shows a flowchart of an Algorithm of Flow.

FIG. 9 shows a CAD drawing of an airlock for sealing an end of a cannula of the present invention. As the screw cap engages the cannula, the flexible insert compresses and creates a seal around objects inserted into the cannula.

FIG. 10 shows a CAD drawing of a satellite-like, umbrella-like, or helical-like mesh configuration for deployment at the end of the fishing catheter of the present invention. If the inner rod is pulled all the way but holding the outer portion the same, a satellite-like mesh structure is formed. If the inner rod stays put but the outer catheter is pushed all the way, an umbrella-like structure will be formed. The mesh wire will form a helical, spiral structure if the inner rod and the middle region are turned in opposite directions.

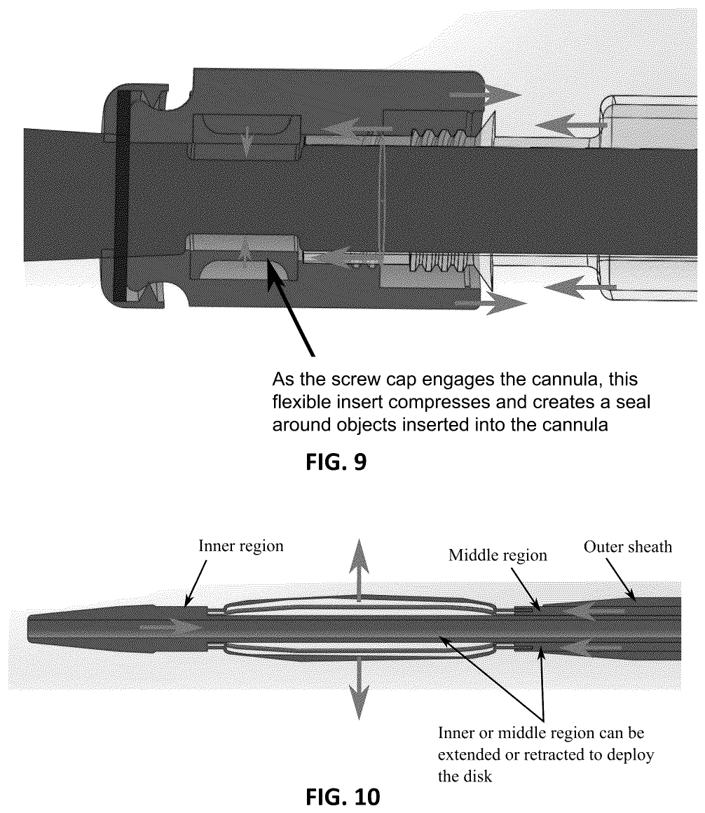

FIG. 11 shows a schematic diagram of a manual pump concept of the present invention which uses a large lockable syringe quick-connect in a T-fashion between two duckbill one-way valves. When the syringe is pulled and locked, it will generate a negative suction to drain blood/clots/unwanted materials through the one-way valve. When it is pushed, the blood/unwanted materials will be pushed through the other one-way valve. The unwanted materials will be filtered and only the de-aired or desired blood is to be returned to the patient. Multiple movable tubing clamps are in place to assist with priming the filter apparatus and to isolate the apparatus in the event of any parts that need to be exchanged. In the absence of the console, the manual pump concept can be used to extract unwanted materials and to return the desired blood. Multiple types of filter manifolds are illustrated.

### DETAILED DESCRIPTION OF THE INVENTION

Following is a list of elements corresponding to a particular element referred to herein:
100 embolism extraction device
120 first/suction catheter
122 first catheter first end
124 first catheter second end
130 reservoir
131 top chamber
132 first filter
133 middle compartment
134 slanted funnel
135 vacuum, suction port
136 bottom chamber slide
137 outlet
140 second/return catheter
142 second catheter first end
144 second catheter second end
150 Fishing catheter
151 Outer sheath aspiration port of the fishing catheter
152 Inner aspiration port of the fishing catheter
153 Fishing rod of the catheter
154 mesh of the fishing catheter
200 console
210 console pedal

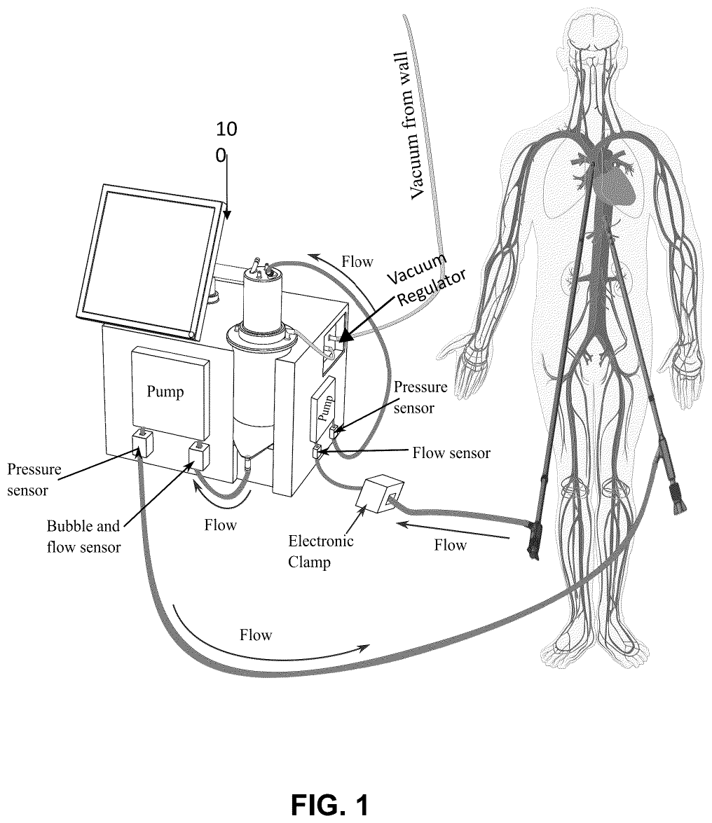

Referring now to FIG. 1, the present invention features a device (100) for extracting unwanted material from a blood vessel. In some embodiments, the device comprises a first catheter (120), a reservoir (130), and a second catheter (140). The first catheter (120) may comprise a first end (122) that is configured to be disposed in a first blood vessel and a second end (124) that is connected to the reservoir (130) through a medical graded commercial available tubing. In some embodiments, the tubing from the second end (124) may be slated into a roller pump to mechanically drain the blood and clot to the reservoir. In another embodiment, the middle chamber of the reservoir is connected to the hospital central vacuum system to mechanically generate negative pressure and drain the blood to the reservoir (130). The reservoir (130) may comprise a top chamber (131), a middle compartment (133), and a bottom chamber (136). In further embodiments, the top chamber (131) may have a first filter (132) disposed therein, and the middle compartment (133) may have a second filter (134) disposed therein. In some embodiments, the middle compartment (133) may be connected to a vacuum pump or a portable pump via a vacuum inlet (135). In other embodiments, the bottom chamber (136) comprises an outlet (137), and the bottom chamber is connected to a roller pump. In some embodiments, the second catheter (140) comprises a first end (142) that is connected to the outlet (137) of the bottom chamber (136), and the second end (144) is configured to be disposed in a second blood vessel via a return cannula.

In some embodiments, the device (100) further comprises a console (200) that regulates the pressure of the vacuum pump and the roller pump. In preferred embodiments, the negative roller pump or vacuum pump controls flow of blood out of a patient and the other roller pump controls flow of blood into a patient. In other embodiments, the console (200) controls a pressure or a flow rate of the device. In other embodiments, the console further comprises a pedal (210) to control the pressure or the flowrate of the device. The negative pump may suction out blood from a patient using a negative pressure (e.g. between about −150 mmHg to −700 mmHg). In preferred embodiments, the pressure used to suction out the blood may be staggered. As a non-limiting example, blood may be continuously or cyclically suctioned from a patient at or up to −700 mmHg to engage the unwanted material. Once the unwanted material is suctioned out with the blood, the negative pressure may be decreased to limit the drainage. Non-limiting examples of the unwanted material include foreign bodies, clots, or other infectious material. In other embodiments, the console (200) is configured to stop negative suction or drainage if a patient's mean arterial blood pressure drops below 65 mmHg. Meanwhile, the console will continue to return blood or crystalloid to restore hemodynamics.

In another preferred embodiment, multiple level sensors (e.g. top, middle, bottom) will be utilized to balance drainage and return as well as safety mechanisms. An algorithm will be implemented into the console to control the drainage and the return of blood. Balancing the flow in the autonomous mode is dependent on the 3 fluid level sensors (at 100 ml, 300 ml, and 800 ml), the vacuum regulator and the return pump. The goal is to balance the fluid/blood at and between the middle and max-level sensors (operating range of 500 ml). For example, drainage still stops (turn off vacuum or drainage pump) when the max fluid level sensor is triggered or true and increase the return pump by 20%. The blood continues to return until the middle level sensor is triggered or true, then drainage resumes. The return pump lowers the Revolutions Per Minute (RPM) by 20%. Return stops when the lowest level sensor is true or triggered or at any given time the bubble sensor is triggered, the entire system will stop. Another option to balance the flow is based on the drainage flow rate and return flow rate. For example, at initiation, the drainage is 2 Liters Per Minute (LPM) at a given negative vacuum pressure, the return pump will increase the RPM to match the return flow of 2 LPM while maintaining fluid in the reservoir between the middle and max-level sensor. If manual mode is activated, autonomous mode will turn off; allowing the user to manually control the drainage and return independently but still abided and governed by the 3 level sensors principles mentioned earlier, and bubble sensors as the final safety check.

In other embodiments, the first blood vessel is a right femoral vein and the second blood vessel is a left femoral vein. In other embodiments, the second blood vessel may be in a different location in the first blood vessel. In some embodiments, the first tube (120) has a length between about 140 cm to 170 cm. In other embodiments, the second tube (140) has a length between about 70 to 100 cm. In preferred embodiments, the first tube (120) may reach the pulmonary artery via the first blood vessel. In other embodiments, the second tube (140) may reach the inferior or superior vena cava. The specific placement of the first tube and the second tube in the pulmonary artery and the vena cava, respectively, works to minimize hemodynamic compromise to the patient. Without wishing to limit the present invention to any theory or mechanism, there are two competing systems in the device: negative and positive pressures. The negative pressure is applied to suction the unwanted material and the positive pressure to deliver blood back to the patient. The opposing pressure negates the procedural effect of the device to the patient's hemodynamics.

In some embodiments, the present invention features a method for removing unwanted material from a blood vessel using any of the devices described herein. The method may comprise: connecting the first end (122) of the first tube (120) to the first blood vessel, connecting the second end (144) of the second tube (140) to the second blood vessel, connecting the device (100) to a console (200), a vacuum pump, and a roller pump, using the console (200) to apply a first pressure on the first tube (120) with the vacuum pump, thereby suctioning blood from the blood vessel to the first tube (120), filtering the blood through the reservoir (130), wherein the unwanted material is filtered from the blood, using the console (200) to apply a second pressure at the bottom of the reservoir (130) using the roller pump, facilitating laminar flow of blood from the reservoir to the second tube (140), and returning the blood to the patient through the second tube (140).

In further embodiments, the console is integrated with computer readable instructions to control the first pressure and the second pressure or a flow rate of the device. In other embodiments, the console may use patient data to build safety measures of the device. Non-limiting examples of the safety measures include if MAP drops 10-30 mmHg or is below 65 mmHg, suction stops, and device delivers fluid/blood slowly until recovery or any number of options to improve safety. Other examples of the use of computer-readable instructions, include but are not limited to, inputting methodology to control volume by looking at RV strain, being slower with volume removal, or to deliver positive fluid balance to reduce the risk of dramatically lowering preload on the left side. In some embodiments the console may have sensors that can stop the device. Non-limiting examples of sensors include pressure sensors, flow sensors, bubble sensors, or level sensors.

Addition of integration with patient monitoring pressures. Determination of the speed of MAP drop to increase outflow before increasing inflow. Thus, if MAP drops 10 mmHg in less than 5 seconds it will reduce drainage and increase return until the MAP increases. Once the bottom level sensor is activated or detected with no volume, it completely stops the return and allows the clinician to decide how to proceed with additional volume or chemical pressure support. It can output a warning as to significant drops in MAP while adjusting the inflow/outflow rates. This will add a layer of safety in addition to making it simpler for the end user.

In some embodiments, the present invention features a device (100) for extracting an embolism from a blood vessel. In some embodiments, the device may comprise a first catheter (120), a reservoir (130), and a second catheter (140). The first catheter (120) may comprise a first end (122) that is configured to be disposed in a first blood vessel and a second end (124) that is connected to the reservoir (130). The reservoir (130) may have a filter disposed therein, and the reservoir may be connected to a vacuum pump and a roller pump. In further embodiments, the reservoir may have an outlet (137) disposed at a bottom end. In some embodiments, the second catheter (140) comprises a first end (142) that is connected to the outlet (137), and the second end (144) is configured to be disposed in a second blood vessel.

Without wishing to limit the present invention to any theory or mechanism, when suction is applied via the vacuum pump, blood containing the embolism is suctioned from the first blood vessel through the first catheter (120) to the reservoir (130). The blood is then filtered through the filter in the reservoir (130) to remove the embolism and air bubbles that may be present in the blood, and the blood is returned to the patient through the second catheter (140).

In some embodiments, the device (100) further comprises a console (200) that regulates the pressure of the vacuum pump and the roller pump. In other embodiments, the console (200) regulates a flowrate of blood being suctioned from a patient and a flowrate of blood being returned to a patient. In other embodiments, the console further comprises a pedal (210) to control the pressure or flowrate of the device. The vacuum pump may suction out blood from a patient using a negative pressure between about −150 mmHg to −700 mmHg. In preferred embodiments, the pressure used to suction out the blood may be staggered. As a non-limiting example, blood may be suctioned from a patient at about −700 mmHg to engage the embolism. Once the unwanted material is suctioned out with the blood, the pressure may be decreased to about 200 mmHg. In further embodiments, the console (200) comprises a sensor that controls the pressure applied to suction out blood.

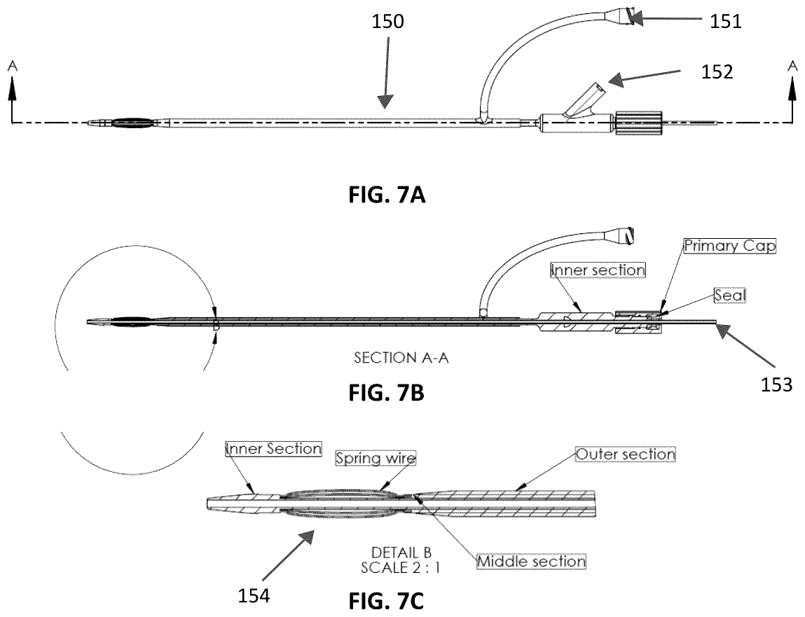

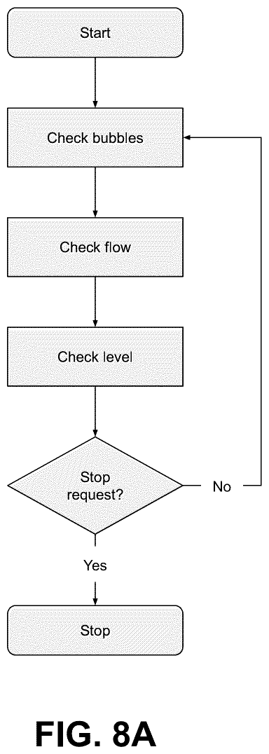

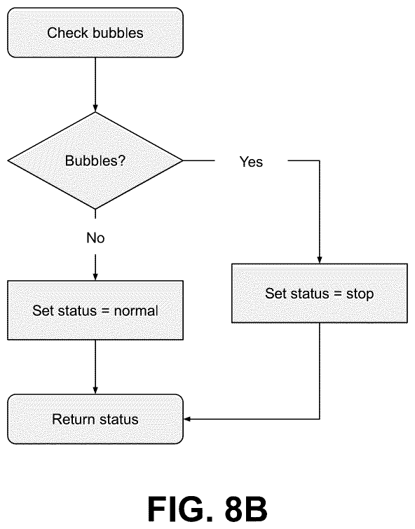

In further embodiments, during suction of the blood from the patient, the reservoir (130) may be opened to visualize the blood. Without wishing to limit the present invention to any theory or mechanism, having a top filter in the reservoir (130) allows an operator of the device to open the reservoir (130) to see if the unwanted material has been filtered out and to send out for pathology. In some embodiments, the device (100) further comprises a daughter catheter (FIGS. 6A-6C) that is deployed from inside the first catheter (120) to reach unwanted materials in smaller vessels. In other embodiments, the device further comprises a screw-in lock cap disposed at the first end of the first catheter (120). In further embodiments, the device (100) further comprises a fishing catheter that is configured to be deployed through the screw-in lock cap to extract the unwanted material toward the first catheter (120). In yet another embodiment, the mesh at the end of the fishing catheter is configured to be expanded when needed and in multiple configurations of a satellite-like, an umbrella-like or a helical-like mesh structure as desired by the end user (FIGS. 7A-7C & 10).

In some embodiments, a roller head pump is attached to a bottom end of the reservoir (130). Without wishing to limit the present invention to any theory or mechanism, the roller head pump prevents blood from being suctioned in the opposite direction in the reservoir (130). The vacuum pump and the roller head pump create competing pressures that allow for the flow of blood in one direction and to prevent hemodynamic compromise in the patient.

In some embodiments, blood may be aspirated from a patient in a volume of up to 1.5 L. In other embodiments, the flowrate of blood being suctioned from a patient may be between about 200 mL/min to 3 L/min. In yet another embodiment, the flowrate of blood being returned to a patient may be between about 200 mL/min to 3 L/min. In some embodiments, the flowrate of the blood being suctioned out of the patient may not be the same as the flowrate of blood going back into the patient.

In other embodiments, the first blood vessel is a right femoral vein and the second blood vessel is a left femoral vein. In some embodiments, the second blood vessel is in a different location in the first blood vessel. In some embodiments, the first catheter (120) has a length between about 140 cm to 170 cm. In other embodiments, the second catheter (140) has a length between about 70 to 100 cm. In preferred embodiments, the first catheter (120) may reach the pulmonary artery. In other embodiments, the second catheter (140) may reach the inferior or superior vena cava. In preferred embodiments, the first filter (132) has a pore size of about 180 μm, and the first filter (134) has a pore size of about 40 μm. In other embodiments, the first filter (132) and the second filter (134) are conical in shape. Without wishing to limit the present invention to any theory or mechanism, the shape of the filters may be important to maintain laminar flow of the blood to eliminate air bubbles that may be in the blood and to minimize hemorrhage and damage.

In preferred embodiments, the first filter (132) has a pore size of about 180 μm, and the second filter (134) has a pore size of about 40 μm. In some embodiments, a shape of the second filter (134) is configured to create laminar flow of blood from the reservoir (130) to the second tube (140). In preferred embodiments, the second filter (134) is conical in shape. In further embodiments, the top chamber (131) is transparent to visualize the unwanted materials and is configured to be opened for sample collection and subsequent pathology. In preferred embodiments, a negative pressure is constantly added to the reservoir to assist in drainage through the filter. In some embodiments, the device (100) further comprises a daughter or fishing catheter that is deployed from inside the first tube (120) to reach unwanted materials in smaller vessels. In further embodiments, a mesh is configured to be deployed through the screw-in lock cap to extract the unwanted material toward the first catheter (120). In yet another embodiment, the mesh is configured to be expanded when needed and in multiple configurations of a satellite-like, an umbrella-like or a helical-like mesh structure as desired by the end user.

In further embodiments, the device may further comprise an inlet disposed on a top end of the reservoir (130). Without wishing to limit the present invention to any theory or mechanism, the inlet may be used to administer a therapeutic composition, crystalloid, or additional blood products to the patient. As a non-limiting example, a spike line may be connected to a port or an inlet on the reservoir, and any necessary therapeutic compositions may be added through the spike line. Examples of therapeutic compositions may include, but are not limited to, anticoagulants, albumin, steroids, vasopressors, or electrolytes.

In other embodiments, the present invention features a method for removing unwanted material from a blood vessel using any of the devices described herein. The method may comprise: connecting the first end (122) of the first catheter (120) to the first blood vessel, connecting the second end (144) of the second catheter (140) to the second blood vessel, connecting the device (100) to a console (200), a vacuum pump, and a roller pump, using the console (200) to apply a first pressure on the first catheter (120) with the vacuum pump, thereby suctioning blood from the blood vessel to the first catheter (120), filtering the blood through the reservoir (130), wherein the unwanted material is filtered from the blood, using the console (200) to apply a second pressure at the bottom of the reservoir (130) using the roller pump, facilitating laminar flow of blood from the reservoir to the second catheter (140), and returning the blood to the patient through the second catheter (140).

In some embodiments, computer readable instructions may be integrated with the console to control the drainage of blood and to return the blood safely back to the patient. In this embodiment, patient data may be fed to the console to build other safety measures beyond basic level sensors or flow sensors. Non-limiting examples of the safety measures include that if MAP drops by 10 mmHg, suction stops and the device delivers fluid/blood slowly until recovery or any number of options to improve safety. Other examples of the use of computer-readable instructions, include but are not limited to, inputting methodology to control volume by looking at RV strain, being slower with volume removal, or to deliver positive fluid balance to reduce the risk of dramatically lowering preload on the left side. In some embodiments the console may be configured to stop the device based on input from one or more sensors. Non-limiting examples of sensors include pressure sensors, flow sensors, bubble sensors or level sensors.

In another embodiment, the present invention features a method for removing unwanted material from a heart or blood vessel. The method may comprise: connecting a first end (122) of a first catheter (120) to a first blood vessel and a second end (124) of the first catheter to a reservoir (130); connecting a second end (144) of a second catheter (140) to a second blood vessel and a first end (142) of the second catheter to the reservoir (130); applying a first pressure on the first catheter (120) via a first pump, thereby suctioning unwanted materials and blood from the heart or blood vessel to the first catheter (120); filtering the blood through the reservoir (130), where the unwanted material is filtered from the blood; applying a second pressure at the bottom of the reservoir (130) using a second pump, thereby facilitating laminar flow of blood from the reservoir to the second catheter (140); and returning the blood to the patient through the second catheter.

In additional embodiments, any of the devices described herein may be used to rapidly transfuse blood or other body fluids to a patient. In this configuration, transfusions can be rapidly performed because the return catheter can be placed near the heart or at the local area where the transfusion needs to take place. Current transfusions require IV, which limits the rate of transfusion and is not close to the heart. In other embodiments, the device may be connected to an oxygenator.

In some embodiments, the console of the present invention may comprise a computing device. The computing device may comprise a processor configured to execute computer readable instructions. The computing device may further comprise a memory component operatively coupled to the processor, comprising computer readable instructions able to be executed by the processor. The computing device may further comprise a communication component operatively coupled to the processor, configured to allow for wired communication, wireless communication, or a combination thereof with one or more sources. The one or more sources may comprise the sensors, an external computing device, or a combination thereof.

In some embodiments, the present invention features a device (100) for extracting unwanted material from a blood vessel. The device may include a suction catheter with a proximal end and a distal end. The distal end may be configured to be disposed in a first blood vessel. The suction catheter may be branched, or may have a single lumen through the catheter. An exterior diameter of the catheter at the distal end may be small enough to fit within the desired blood vessel. Alternatively, for example if the catheter is too big to fit within the desired blood vessel, a smaller tapered daughter catheter may be passed through the suction catheter to fit within the desired blood vessel. The suction catheter may be attached to one or more pumps so as to generate a negative pressure for suctioning unwanted materials and blood from the blood vessel. In preferred embodiments, the entire lumen of the suction catheter has a large enough diameter to suction the unwanted material from the blood vessel without clogging the catheter. Furthermore, in branched suction catheters with the suction port disposed to the side of the catheter, the sweeping path may be at an angle designed to allow suctioning of the unwanted material from the blood vessel without clogging the catheter.

The device may include a return catheter comprising a proximal end and a distal end. The distal end may be configured to be disposed in a second blood vessel, for example, to return filtered blood suctioned from the first blood vessel to the second blood vessel. Just as the suction catheter may be branched or may have a single lumen through the catheter, the return catheter may be branched or may have a single lumen.

The device may include a flow system disposed between the suction catheter and the return catheter and fluidly coupled with the proximal ends of both catheters, wherein the flow system is configured to induce a suction flow from the suction catheter and a return flow to the return catheter. This flow system may include any suitable combination of pumps. As a non-limiting example, the flow system may include a suction pump, a fluidic connection with a vacuum system, and a return pump. In some embodiments, the suction pump and the return pump may each be roller pumps. In other embodiments, the flow system may include one or more manual pumps such as manually actuated syringes.

The device may also include a filter system disposed between the suction catheter and the return catheter and fluidly coupled with the proximal ends of both catheters, wherein the filter system is configured to capture unwanted material extracted from the blood vessel via the suction flow such that it is not returned to the second blood vessel via the return flow. As a non-limiting example, the filter system may include a filter reservoir having: a top chamber having a first filter disposed therein, the top chamber comprising an inlet which is fluidly coupled with the suction catheter; a bottom chamber comprising an outlet which is fluidly coupled with the return catheter, and a vacuum inlet fluidly coupled with a vacuum source; and a middle compartment disposed within the bottom chamber, the middle compartment having a second filter disposed therein. In some embodiments, the second filter comprises a funnel or cone-like structure. Without wishing to limit the present invention to any particular theory or mechanism, it is believed that the shape of the second filter may promote laminar flow, thereby reducing the possibility of introducing air bubbles into the return flow. In some embodiments, a pore size of the first filter is less than about 100, 110, 120, 130, 140, 150, 160, 170, 180, 190, 200, 220, 240, 260, 280, or 300 μm and a pore size of the second filter is less than about 10, 20, 30, 40, 50, 60, 70, 80, 90, or 100 μm.

According to preferred embodiments, the flow rates of the suction flow and the return flow may be independently adjusted. As a non-limiting example the suction and return pumps may operate at different speeds in order to provide a suction flow that is greater to, equal to, or less than the return flow. The device may additionally include a console configured to control pressure or flow rates of the suction flow and the return flow based on input from a plurality of sensors. Furthermore, the console may be configured to stop the suction flow, the return flow, or both if a potentially unsafe condition is predicted based on input from the plurality of sensors. As non-limiting examples, the plurality of sensors may include flow sensors, bubble sensors, pressure sensors, fluid level sensors, or a combination thereof.

In preferred embodiments, the device is configured to extract the unwanted material without significant overall loss of blood. As non-limiting examples, the device may be configured to extract the unwanted material with overall blood loss of less than about 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 60, 70, 80, 90, 100, 120, 140, 160, 180, 200, 250, 300, 350, 400, 450, 500, 600, 700, 800, 900, or 1000 cc of blood.

The flow system may induce a pulsed flow. As a non-limiting example, a flow rate or the speed of the operating pump may be increased and decreased cyclically to induce a vibration/sonication effect to assist in the extraction of unwanted materials. In some embodiments, the suction catheter and the return catheter each comprise a through-pathway configured for allowing implements to access through the catheter. As a non-limiting example, the through-pathway may provide for the application of the daughter catheter or the fishing catheter in the suction catheter in order to reach smaller spaces/cavities.

In some embodiments, the present invention features a suction catheter for extracting unwanted material from a blood vessel. As a non-limiting example, the catheter may include: a catheter body comprising a proximal end and a distal end, wherein the distal end is configured to be disposed in the blood vessel; a distal opening in the distal end of the catheter body; a suction port and an access port branching from the proximal end of the catheter body, wherein the suction port is configured to be fluidly coupled with a flow system configured to induce a suction flow from the suction catheter, and wherein the access port is configured to allow access through the catheter to the distal opening; and an air-lock coupled with the access port, wherein the air-lock is configured to seal itself or seal around an implement inserted through the access port. In preferred embodiments, a pathway from the distal opening to the suction port has a minimum diameter of at least about 1, 2, 3, 4, 5, 6, 7, 8, 9, 10, 11, 12, 13, 14, 15, 16, 17, 18, 19, or 20 mm. The implement may be a fishing catheter configured to be deployed through the catheter body to reach unwanted material from smaller blood vessels. The implement may be an expandable satellite-like, an umbrella-like, a helical-like mesh structure configured to be deployed through the catheter body to extract unwanted material toward the distal opening.

In some embodiments, the present invention may feature a method for removing unwanted material from a blood vessel. As a non-limiting example, the method may include: providing a device having a suction catheter, a return catheter, and flow and filter systems disposed between the suction catheter and the return catheter; inserting a distal end of the suction catheter into a first blood vessel; inserting a distal end of the return catheter into a second blood vessel; actuating the flow system so as to induce a suction flow from the suction catheter and a return flow to the return catheter; and using the filter system to filter blood removed from the first blood vessel via the suction flow prior to reintroducing it to the second blood vessel via the return catheter, thereby removing the unwanted material. In some embodiments, the first or second blood vessel may be a left or right femoral vein. As a non-limiting example, the first blood vessel may be a left femoral vein and the second blood vessel may be a right femoral vein.

### EXAMPLES

The following are non-limiting examples of the present invention. It is to be understood that said examples are not intended to limit the present invention in any way. Equivalents or substitutes are within the scope of the present invention.

### Example 1: Computer Assisted Extraction of Unwanted Materials and Return of Desired Blood

### Description of Suction Catheter

The total length of the suction catheter is 145 cm. From the distal tip to the 30 cm mark, the catheter has variable diameters (24, 22, and 20 Fr). The thickness of the catheter is about 0.1 mm with a flexible spiral metal inside that is 3 mm apart per revolution. The distal tip of the catheter has 2 mm soft atraumatic soft material. After the 30 cm mark, the diameter is 24 Fr up to the locking mechanism. The thickness of the catheter is 0.1 mm with a flexible spiral metal insert that is 1 mm apart per revolution. The catheter has a screw-in locking mechanism that is 6 cm in length. The side port for negative suction or return is 9.5 mm. If steerability is needed a catheter with a rotating knob is also available.

There is a locking mechanism (air-lock) at the proximal end of the catheter: the cap screws into the catheter and as the cap is tightened, the rubber material inside the cap is compressed, thereby occluding the lumen. This mechanism prevents air from getting into the system during aspiration. At the end of the cap there is a rubber membrane that prevents blood from leaking.

The dilator of the catheter is 159 cm in length and tracks through a 0.35 inch standard stiff wire. The dilator is made of plastic and the distal tip of the dilator is tapered and smooth. It also has a tight fit with the distal segment of the catheter. The size of the dilator will depend on the distal catheter size. For DVT/small vessel clots, a 12, 10, 8, and 6 Fr size catheter can be used.

### Description of the Tapered Daughter Catheter

The length of the tapered daughter catheter is 40 cm, and the catheter size can be tapered 18, 16, 14, 12, or 10 Fr. The daughter catheter has a metal rod connector that is 126 cm. The distal tip of the catheter has a ball connector. The dilator length is 160 cm and a recessed canal to fit the metal rod so that when merged together, they can fit inside the suction catheter The end of the daughter catheter and dilator is blunted and smoothened.

### Description of the Fishing Catheter (FIGS. 7A-7C & 10)

The total length of the dilator is 166 cm and the fishing catheter length (150) is about 182 cm. The mesh (154) length is about 30 mm. The screw-in lock caplock mechanism is 4.6 cm long.

The mesh (154) is made out of 8 moldable resilient metal wires like nitinol that are about 1 mm in thickness, 1 mm width×30 mm in length. When open, the total diameter of the mesh (154) is 18 mm. The 8 wires are attached distally to the dilator and proximally to a sliding catheter. The mesh (154) is connected to the said catheter at a 30-degree angle or vice versa (30-degree at the tip of the dilator). This angulation will then cause the mesh (154) to rotate and form a helical shape. During insertion to the body, that mesh (154) is retracted and is covered by a sheath (151). Once inserted inside the vessel, the sheath (151) and the inner region of the fishing catheter (152) is de-aired. The first part of mesh (154) deployment is to pull the sheath to uncover the retracted mesh (154). The second part is pushing the delivery catheter forward to the direction of the tip of the dilator in a circular fashion. This will be marked by 90, 180, 270, 360 degrees. The size of the mesh (154) is dependent on the forward position of the catheter. if the inner rod (153) is pulled while the middle region is locked, the mesh can form a satellite-like mesh structure. If the inner rod (153) is locked but the middle region is pushed forward, the mesh can form an umbrella-like mesh structure. The size will also be marked relative to the degree of rotation. After the desired mesh (154) size and structure is achieved, the locking mechanism can then be tightened to secure the mesh (154) size and the position.

Locking the catheter will prevent the mesh (154) from collapsing when the system is pulled for clot extraction. The mesh (154) with captured clots will be pulled toward the suction (long) catheter.

### Description of Console Algorithms

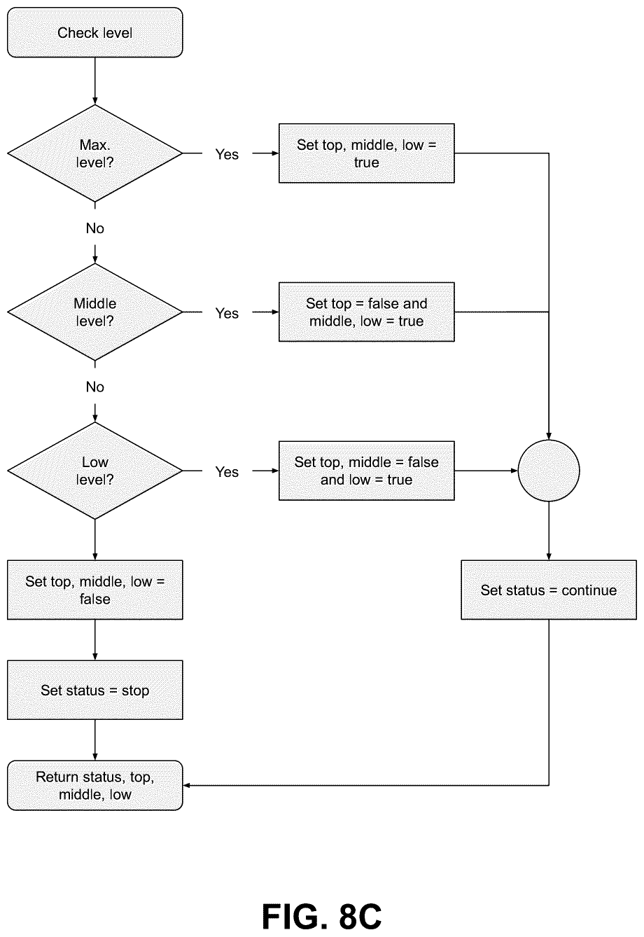

Referring now to FIG. 8A, the present invention features an algorithm of system check at initiation. The system checks for the presence of bubbles sensor, flow sensors in place, and fluid level engagement.

Referring now to FIG. 8B, the present invention features an algorithm for checking bubbles: This subroutine is waiting for a signal from a sensor that indicates an air bubble has been detected. If not, the operation continues as normal. If a bubble is detected all operation stops. The electronic clamp will engage to prevent further suctioning of the blood.

Referring now to FIG. 8C, the present invention features an algorithm for checking fluid levels. This subroutine checks the level of the fluid in the reservoir. It requires at least two sensors that are positioned at specific locations near the top (max) and bottom of the reservoir. It first checks to see if there is fluid at or above the max level sensor, in which case all conditions are returned as true. If not, it checks the middle position (if available), in which case it returns the max position as false and the middle and bottom position as true. If not again, then it returns the max and middle positions as false and the bottom position as true.

Referring now to FIG. 8D, the present invention features a console configured to execute a flow check algorithm. This subroutine checks the flow rate of the fluid being removed and returned. First it checks if the flow rates are equal. If yes, it returns a normal status. If not, then it checks if the console is in manual control mode. If yes, the status is returned as normal. If not in manual control mode, then it calls the Balance flow subroutine. It then checks the flow rate again. If it is equal then status is returned as normal. If not, the operation is stopped. For balance flow, it is dependent on the 3 fluid level sensors (at 100 ml, 300 ml, and 800 ml), the vacuum regulator and the return pump. The goal is to balance the fluid/blood at and between the middle and max-level sensors (operating range of 500 ml). For example, drainage still stops (turn off vacuum or drainage pump) when the max fluid level sensor is triggered or true and increase the return pump by 20%. The blood will continue to return until the middle level sensor is triggered or true, then drainage will resume. The return pump lowers RPM by 20%. Return stops when the lowest level sensor is true or triggered or at any given time the bubble sensor is triggered, the entire system will stop. Another option to balance the flow is based on the drainage flow rate and return flow rate. For example, at initiation, the drainage is 2 LPM at a given negative vacuum pressure, the return pump will increase the RPM to match the return flow of 2 LPM while maintaining fluid in the reservoir between the middle and max-level sensor. If manual mode is activated, autonomous mode will turn off; allowing the user to manually control the drainage and return independently but still abided and governed by the 3 level sensors principles mentioned earlier, and bubble sensors as the final safety check.

### Description of the Device

The present invention features a device for extracting unwanted material comprising clots in a pulmonary artery. In some embodiments, the device may comprise a suction catheter comprising a proximal end and a distal end. The distal end may be configured to be disposed in a first blood vessel. The device may further comprise a return catheter comprising a proximal end and a distal end. The distal end may be configured to be disposed in a second blood vessel. The device may further comprise a flow system disposed between the suction catheter and the return catheter and fluidly coupled with the proximal ends of both catheters. The flow system may be configured to induce a suction flow from the suction catheter and a return flow to the return catheter.

The device may further comprise a two stage filter system disposed between the suction catheter and the return catheter and fluidly coupled with the proximal ends of both catheters. The filter system may comprise a first stage filter comprising a conical pyramid structure configured to enhance visibility of the clots. The filter system may further comprise a second stage filter comprising a housing, a solid funnel structure disposed within the housing, and a half-walled sliding spill plate disposed below the solid funnel structure. The filter system may further comprise a slope structure disposed below the second stage filter. The filter system may be configured to capture unwanted material in blood extracted from the first blood vessel via the suction flow such that the unwanted material is captured on the first stage filter and blood is further filtered in the second stage. The spill plate may comprise a 45 degree sloped shape on a first half and a wall having a height of 1 cm on a second half opposite the first half such that laminar flow is achieved in the blood in one direction as the blood passes through the solid funnel structure into the spill plate and onto the slope disposed at a bottom of the filter system. Only filtered and de-aired blood may be returned via the return catheter.

In some embodiments, the flow system may comprise a suction pump, a fluidic connection with a vacuum system, and a return pump. In some embodiments, the suction pump and the return pump may each comprise roller pumps. In some embodiments, the suction flow system may comprise a manually actuated 100 ml syringe pump with 2 one way valves before and after the syringe pump in lieu of suction pump or vacuum pump. The syringe pump may comprise a large bore quick-connect port. The syringe pump may be configured to lock when fully pulled to generate negative pressure. The syringe pump may be configured to move clots and blood from the suction catheter to the filter system.

In some embodiments, the filter system may further comprise a filter reservoir. The reservoir may comprise a top chamber comprising a first conical pyramid filter disposed therein and an inlet fluidly coupled with the suction catheter. The reservoir may further comprise a bottom chamber comprising an outlet fluidly coupled with the return catheter and a vacuum inlet fluidly coupled with a vacuum source. The reservoir may further comprise a middle compartment disposed within the bottom chamber, the middle compartment comprising a second filter disposed therein. The reservoir may further comprise a hardshell reservoir configured to withstand high negative pressure, comprising a pressure release valve configured to release at −760 mmHg or less. The top chamber may be removable to access unwanted materials.

In some embodiments, the second filter may comprise a cylindrical funnel structure connecting to the first filter to generate laminar blood flow toward the spill plate to de-air blood. In some embodiments, the pore size of the first filter may be about 180 μm and the pore size of the second filter is about 40 μm. In some embodiments, flow rates of the suction flow and the return flow may be configured to be independently adjusted. In some embodiments, the device may be configured to extract the unwanted material without significant overall loss of blood. In some embodiments, the device may be configured to rapidly infuse a volume of blood. In some embodiments, the suction catheter and the return catheter may each comprise a through-pathway configured for allowing implements to access through the catheter.

The present invention features a suction catheter for extracting unwanted material from blood extracted from a blood vessel. The catheter may comprise a catheter body comprising a proximal end and a distal end. The distal end may be configured to be disposed in the blood vessel. The proximal end may be configured to be fluidly coupled to a filter system comprising a solid funnel structure disposed within and a half-walled sliding spill plate disposed below the solid funnel structure. The spill plate may have a sloped shape on a first end and a wall at a second end opposite the first end such that laminar flow is achieved in the blood in one direction as the blood passes through the solid funnel structure into the spill plate and onto a slope disposed at a bottom of the filter system.

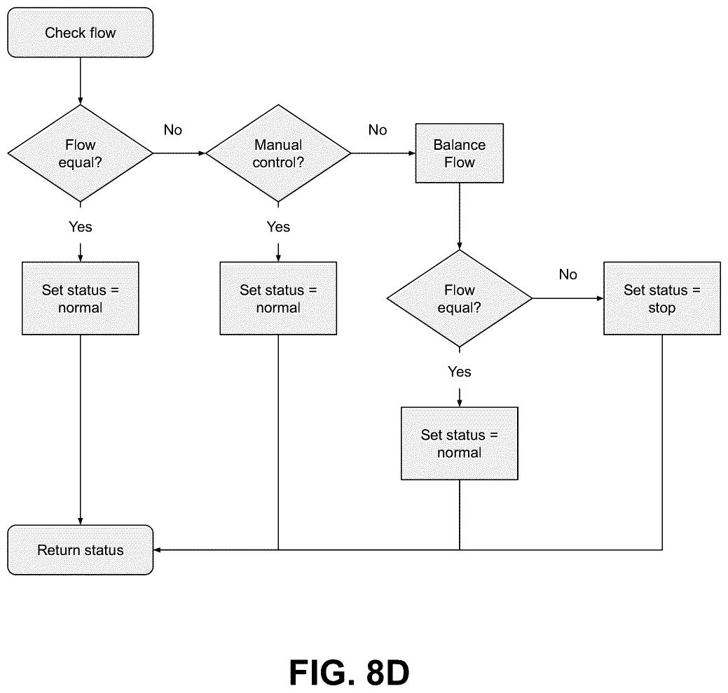

The catheter may further comprise a distal opening in the distal end of the catheter body comprising a screw cap end containing a flexible rubberized insert configured to compress inserted objects (FIG. 9). The catheter may further comprise a side suction port and an access port branching from the proximal end of the catheter body. The side suction port may be configured to be fluidly coupled with a flow system configured to induce a suction flow of the blood from the suction catheter. The access port may be configured to allow access through the catheter to the distal opening. The catheter may further comprise an air-lock coupled with the access port. The air-lock may be configured to seal itself or seal around an implement inserted through the access port. The catheter operatively coupled to the filter system may be configured to be controlled by a plurality of sensors configured to monitor negative vacuum pressure in the filter system, a flow rate of the blood into the filter system, and a fluid level of the blood in the filter system, and a console operatively coupled to the filter system and the plurality of sensors, configured to execute computer readable instructions for controlling pressure or flow rate of the suction flow in the filter system such that the suction flow is stopped by the console when bubbles are detected or when the fluid level is above a maximum threshold, increasing the suction flow when the fluid level is below a minimum threshold, or a combination thereof. In some embodiments, a pathway from the distal opening to the suction port has a minimum diameter of at least about 9 mm. In some embodiments, the implement may comprise a daughter catheter configured to be deployed through the catheter body to reach unwanted material from smaller blood vessels.

The present invention features a fishing catheter configured to be deployed through a delivery catheter disposed in a vessel, configured to extract unwanted material toward the suction catheter. The catheter may comprise an expandable mesh comprising 8 moldable resilient metal nitinol wires. Each wire may be about 1 mm in thickness, 1 mm in width, and 30 mm in length. The expandable mesh may be configured to shift into an open configuration upon actuation. A diameter of the mesh in the open configuration may be 18 mm. The catheter may further comprise a dilator comprising a distal end coupled to the 8 wires. The catheter may further comprise a sliding catheter comprising a proximal end coupled to the 8 wires. The 8 wires may be coupled to the dilator, the sliding catheter, or a combination thereof at a 30-degree angle such that the mesh rotates to form a helical circular disk.

The catheter may further comprise a sheath component coupled to the mesh, configured to cover the mesh as the mesh is retracted during insertion into the vessel. The sheath may be de-aired upon insertion into the vessel. The mesh may be configured to deploy by a deployment process. The deployment process may comprise pulling the sheath to uncover the mesh and pushing the delivery catheter forward towards the distal end of the dilator in a circular fashion. The mesh may comprise a satellite-shape mesh structure, an umbrella-shape mesh structure, or a helical-shape structure.

The present invention features a method for removing unwanted material from a left or right femoral blood vessel. The method may comprise providing a device comprising a suction catheter, a return catheter, a flow system disposed between the suction catheter and the return catheter, and a filter system disposed between the suction catheter and the return catheter, the filter system comprising a solid funnel structure disposed within and a spill plate disposed below the filter system. The spill plate may have a sloped shape on a first end and a wall at a second end opposite the first end such that laminar flow is achieved in the blood in one direction as the blood passes through the solid funnel structure into the spill plate and onto a slope disposed at a bottom of the filter system. The device may further comprise a plurality of sensors operatively coupled to the filter system and a console operatively coupled to the filter system and the plurality of sensors.

The method may further comprise inserting a distal end of the suction catheter into a first blood vessel. The method may further comprise inserting a distal end of the return catheter into a second blood vessel. The method may further comprise actuating the flow system so as to induce a suction flow from the suction catheter and a return flow to the return catheter. The method may further comprise using the filter system to filter the blood removed from the first blood vessel via the suction flow prior to reintroducing it to the second blood vessel via the return catheter, thereby removing the unwanted material. The method may further comprise measuring, by the plurality of sensors, negative vacuum pressure in the filter system, a flow rate of the blood into the filter system, and a fluid level of the blood in the filter system. The method may further comprise stopping, by the console, the suction flow and the return flow when bubbles are detected, when the fluid level is below a threshold, or a combination thereof.

The present invention features a device for extracting unwanted material from a blood vessel. The device may comprise a suction catheter comprising a proximal end and a distal end. The distal end may be configured to be disposed in a first blood vessel. The device may further comprise a return catheter comprising a proximal end and a distal end. The distal end may be configured to be disposed in a second blood vessel. The device may further comprise a flow system disposed between the suction catheter and the return catheter and fluidly coupled with the proximal ends of both catheters. The flow system may be configured to induce a suction flow from the suction catheter and a return flow to the return catheter.

The device may further comprise a filter system disposed between the suction catheter and the return catheter and fluidly coupled with the proximal ends of both catheters, the filter system comprising a solid funnel structure disposed within, and a spill plate disposed below the solid funnel structure. The filter system may be configured to capture unwanted material in blood extracted from the blood vessel via the suction flow such that the unwanted material is not returned to the second blood vessel via the return flow. The spill plate may have a sloped shape on a first end and a wall at a second end opposite the first end such that laminar flow is achieved in the blood in one direction as the blood passes through the solid funnel structure into the spill plate and onto a slope disposed at a bottom of the filter system. The device may further comprise a plurality of sensors operatively coupled to the filter system, configured to measure negative pressure in the filter system, a flow rate of the suction flow and a flow rate of the return flow, and a fluid level of the blood in the filter system.

The device may further comprise a console operatively coupled to the filter system and the plurality of sensors, comprising a processor configured to execute computer readable instructions and a memory component operatively coupled to the processor, comprising computer-readable instructions. The computer-readable instructions may comprise intaking, by the plurality of sensors, data on the suction flow, the return flow, reservoir weight, bubble presence, level sensors comprising a lowest level sensor, a middle level sensor, and a highest level sensor, and patient parameters. The computer-readable instructions may further comprise adjusting thresholds of the data in real-time tailoring to a condition or sequence of the procedure. The computer-readable instructions may further comprise detecting, by the plurality of sensors, whether or not bubbles are present in the filter system and whether or not the lowest level sensor is appropriate to engage. The computer-readable instructions may further comprise stopping, if the bubbles are detected or the lowest level sensor is appropriate to engage, the suction flow and the return flow. The computer-readable instructions may further comprise determining, by the plurality of sensors, whether the flow rate of the suction flow is equal to the flow rate of the return flow. The computer-readable instructions may further comprise adjusting, if the flow rate of the suction flow is not equal to the flow rate of the return flow, the flow rate of the suction flow and the flow rate of the return flow such that the flow rate of the suction flow is equal to the return flow. The computer-readable instructions may further comprise simultaneously adjusting, if the flow rate of the suction flow is not equal to the flow rate of the return flow, the return flow and a vacuum for suction flow. The computer-readable instructions may further comprise determining, by the plurality of sensors, the fluid level within the filter system. The computer-readable instructions may further comprise stopping, if the fluid level within the filter system is below a fluid level threshold, the suction flow and the return flow. The computer-readable instructions may further comprise intaking and assessing, if patient parameters require more volume. The computer-readable instructions may further comprise adjusting the suction flow and return flow to restore patient hemodynamics.

### Description of the Return Cannula

The total length of the return cannula is 35 to 70 cm, from the proximal end to the distal end. The diameter of the return cannula can be 19 Fr, 17 Fr, or 15 Fr. The tip has a 3 mm flexible/soft thin material, followed by 4 sets of 3 laser cut holes (12 holes total), each separated by 1 cm. The hole diameter is about 3 mm. From the tip to the first hole is 7 mm long. From the first hole to the suture lock is about 21 cm that will be reinforced with mesh metal just like the return cannula. From the suture lock to detail A is about 4 cm of just clear plastic for clamping. The screw-in lock cap is exactly as described in the long suction cannula. The dilator is 47 to 77 cm long and will be locked down with the screw-in lock cap (detail B) just as described in the long suction cannula.

### Description of the Reservoir

The reservoir has plastic connectors at the base for blood delivery, and at the top for negative suction of blood and clots (these are 9.5 mm connectors). The suction connector is connected to the thrombectomy device and the delivery connector is connected to the return cannula using a 9.5 mm medical grade (e.g. Tygon) tube. The top-hat-shaped canister houses the top filter which is an inverted V that is 9.2 cm in height and is covered with 180-micron filter media to separate the aspirated thrombus from the blood. There are also two standard Luer lock connectors for delivering blood products and crystalloids to the system. In production, this top hat canister is screwed air tight into the main reservoir. A rubber O ring will ensure a complete seal between the top hat component and the main reservoir. There are two connectors at the bottom part of the top hat. One is for connection to the suction regulator and the other is a standard Luer lock. The reservoir is connected to the suction regulator using medical grade tubing. Inside the main reservoir is the main filter. The main filter comprises an outer circular plastic component with 4 support ribs for structural stability. A 40 micron filter is sandwiched between this structure and the v-shaped inside funnel. The V-shape cylindrical funnel assists with laminar blood flow and prevents microbubble formation. A half-walled spill plate is at the bottom of this structure for support and to promote laminar blood flow to the bottom processed blood containment section. This section acts as a de-airing chamber and can accommodate 270 cc of volume. This section will be full of saline during device setup since the reservoir will be primed with 500 cc of normal saline.

This blood containment section also has multiple level sensors. The top sensor is at the 800 mL mark and the bottom is at the 100 mL. When the device is turned on to suction blood, the device will first give a predetermined amount (e.g. 200 cc) of fluid to the patient before the negative pressure is turned on. This increase in additional volume to the patient will increase preload and will mitigate the possible hemodynamic decline as the device aspirates blood from the pulmonary artery. Once fluid volume is achieved the suction will be turned on. The flow of blood in the negative suction catheter will be equal or close to the positive flow of blood to the return cannula which is blood going back to the patient. The device will stop when the amount of blood is only 100 mL.

The bottom of the reservoir has a 9.5 mm connector with three barbs. This is connected to the return cannula using a 9.5 mm medical grade tube. The entire assembly will be approximately 38.4 cm.

### Description of the Console

The console will generate negative force to suction the foreign materials along with blood from the patient into a reservoir; where blood and foreign materials are filtered and separated so that blood from the reservoir can be pumped back to the patient through a peristaltic pump with 9.5 mm″ medical grade tubing, multiple level sensors, bubble sensors, pressure sensors and flow sensors.

From the long suction cannula, a 9.5 mm inch medical grade tubing will be connected at the 9.5 mm sideport of the cannula to the top of the reservoir. Once the long cannula is situated appropriately at the clot, a peristaltic inflow pump will begin generating negative force with an RPM rate of 30 Mechanical suction at −20 mmHg will be applied to the reservoir to avoid positive pressure and assist in filter drainage via a regulator to the hospital suction source. The outflow pump will match the RPMs of the inflow pump to immediately balance volume. The higher the RPMs of the inflow pump, the higher the suction flow. There will be (multiple) level sensors embedded into the console at the back of the reservoir. The lowest will be placed at approximately 100 mL to start the transfusion at 30 RPM. If the lowest sensor is activated, it will stop the forward pump. The highest level will be at 800 mL to increase the RPM by 100% (2+LPM) but also stop the suction roller if it gets activated. Middle level sensors will balance flow appropriately. A drainage flow sensor and return flow sensor may also be present. The console may be configured to execute computer-readable instructions for matching the flow rate. The console will also have a bubble sensor prior to the outflow pump head, acting as a safety mechanism to stop the return flow. There will be a one way valve in the tubing between the reservoir and the pump head to minimize gaseous micro-embolism due to high vacuum and cavitation. The peristaltic pump can be disengaged within the console or foot pedal and vacuum can be applied up to −700 mmHg from a hospital source. The peristaltic pump can be engaged to stop the vacuum source and stop all flow.

There will be a pressure sensor with flow sensor integrated distal to the pump head to match the flow rate of the drainage but also act as a safety mechanism to stop the pump head if post pressure is above 350 mmHg.

The vacuum source is from the hospital wall, which can be supplied up to −700 mmHg. The console can act as a suction device using mechanical suction if the tubing is not loaded into the inflow pump side. There will be a regulator inside the console that will interface with the lowest sensor and will be controlled automatically or manually by the end user and with a joystick at the field. The pump can be engaged or disengaged via the console or foot pedal to allow for vacuum to be applied to the line.

The console will be turned on and off with the pedal with an OFF override button on the pedal. Safety mechanisms that will stop the pump include bubble sensor, pressure sensors and level detectors.

### Using the Device

Access is via the right and left femoral vein. The suction cannula is located on the right and the return cannula will be on the left. Access will be done via ultrasound guidance and a micropuncture 6 Fr system. A stiff 0.35 inch wire is then inserted to the 6 Fr sheath. The right 6 Fr sheath is then replaced with a 24 Fr sheath. The said sheath is sutured in place. Heparin should be given at 100 μg per kg. The left sheath is replaced over a 0.35 inch wire with the return cannula. A right heart catheter is then inserted inside the return cannula and is floated to the pulmonary artery. Over a 0.35 inch×280 cm in length wire the right heart catheter is removed and replaced with a 6 Fr pigtail catheter. The pigtail is then connected to a manifold or assist device for continuous pressure measurement and for angiography of the respected pulmonary artery. ACT should be checked after 5 mins of administration and should be >300. The pulmonary artery with the biggest clot burden should be treated first. A right heart catheter is inserted to the 24 Fr sheath and is floated to the target pulmonary artery. A 0.35 curve hydrophilic wire is inserted to the right heart catheter while the balloon is inflated, and the pulmonary artery is wired. After proper wire position is achieved distally, the right heart catheter is inserted distally for wire exchange. The curve hydrophilic wire is removed and a 1 cm stiff Amplatz guidewire is inserted to the target artery. The suction catheter and the return catheter are then connected to the console using the 9.5 mm tubing.

The console houses the reservoir for blood filtering and delivery. The reservoir is connected to the negative pressure regulator in the console. The negative pressure regulator is then connected to the hospital negative pressure wall system. The reservoir is then connected with a peristaltic pump to the thrombectomy device (suction catheter) using a 9.5 mm medical grade tube. Flow will be monitored using a flow sensor that is located just before the tube connects to the reservoir. The right heart catheter is removed over the wire and the desired thrombectomy catheter is inserted to the pulmonary artery. The negative pressure will start at a negative 100 mmHg or 30 RPMs and will increase to negative 600 mmHg or 150 RPMs if there is less than 100 mL/min of blood flow. If blood flow exceeds 500 mL/min then negative pressure will decrease to 100 mmHg or 10 RPMs. A peristaltic pump will deliver blood, fluids or medications back to the patient while suctioning and filtering blood.

### Example 2: Manual Extraction of Unwanted Materials and Return of Filtered Blood

In an embodiment without a console, the present invention may feature a manual device for removing unwanted material from a heart or blood vessel and returning the desired blood back to the patient. The method for using said device may comprise: disposing a first end (122) of a first catheter (120) within a first blood vessel and connecting a second end (124) of the first catheter to a first duckbill-like one-way valve via a 9.5 mm diameter medical grade tubing. A tubing clamp will be present prior to the first one-way valve to clamp the tubing line to prevent bleeding or air. The first one-way valve will then be connected to a T-junction connector then to the second one-way valve and subsequently to the filter of the present invention. The filter is then connected to the second end (144) of a second catheter (140) to a second blood vessel so that extracted blood can be returned to the patient. Another tubing clamp will be present between the filter and the second catheter to stop bleeding or transfusing any unwanted materials or allowing filter exchange. In the absence of the console, another manual syringe pump system with one way valves can be added prior to the return cannula to transfuse the desired blood.

In another embodiment, the filter apparatus/manifold is consisted of a 9.5 mm inlet at the top side, middle, or bottom; and a 9.5 mm outlet at the bottom side with a 40-120 micron filter in multiple potential configurations to filter out foreign materials; and a luer port at the very top to de-air or prime with crystalloid; and can hold 300-500 mL of blood. A 100 mL or larger lockable syringe may be quick-connected to the T-junction with the presence of another tubing clamp on the tubing between the syringe and the T-junction. These one-way valves will direct blood flow from the suction cannula to the filter then back to the return cannula through the pull and push of the syringe. The filter can be configured to fill from the bottom and flow through a step to the filter and back down to a bottom outlet, or the filter can fill from the top portion and pass through a filter to exit out of the bottom in various shapes allowing it to rest on the operative field and reduce the likelihood of air embolism to the patient. All ports, connectors, and tubing are 9.5 mm or ⅜ inches in diameter.

In another manual extraction embodiment, the setup for extracting unwanted materials could be reconfigured to include in sequence of: 1) the extraction cannula followed by tubing w/tubing clamp, followed by the 2) first one-way valve, which is connected to the 3) present filter apparatus via tubing, which then connected to the T-junction connector that has the 100 ml syringe; then connected to the second one-way valve which is then connected the return cannula with a tubing clamp in the middle.

### Using the Manual Extraction Device

As a non-limiting example of use of the extraction device, the clinician places the suction cannula in the heart or pulmonary artery as described. The syringe is filled with 100 mL of normal saline and used to de-air the tubing before connecting it to the return cannula. Once the entire apparatus is de-aired or primed with crystalloid, The clinician will close the tubing clamp #1 and #3 in anticipation of hooking up to the extraction and return cannula. The clinician will engage a clot, wet-to-wet connect the cannula, and keep the tubing clamp closed. The 100 ml syringe will be pulled to generate negative pressure and lock it in place. If more negative is desired, the clinician can close the tubing clamp #2, which is right distal to the syringe and before the T-Junction connection; disconnect the syringe to reload it then quick-connect it back to the T-junction to pull again to generate more negative pressure. This step can be repeated as desired by the clinician. Once a negative suction is achieved within the syringe and the system, clinician will release tubing clamp #1 and #2. Once the syringe is filled, it can be pressed to reinfuse the blood because the one way valves will ensure proper flow from the suction cannula through the filter to the return cannula. This approach can be repeated until all or most unwanted materials are removed. The clinician can also choose to use a sheath or any available cannula (in lieu of the return cannula) within their practice that's deemed appropriate to infuse blood or crystalloid. If the filter is fully clotted; it can be exchanged through existing connections. After the completion of the procedure, normal saline can again be loaded into the syringe to displace the residual blood in the circuit so that blood can be given back to the patient.

### Further Description of the Console and the Computer Readable Instructions

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Enumerated Embodiments: The following are non-limiting examples of enumerated embodiments.

Enumerated Embodiment 1: A device (100) for extracting unwanted material from a blood vessel, the device comprising:
a. a suction catheter comprising a proximal end and a distal end, wherein the distal end is configured to be disposed in a first blood vessel;
b. a return catheter comprising a proximal end and a distal end, therein the distal end is configured to be disposed in a second blood vessel;
c. a flow system disposed between the suction catheter and the return catheter and fluidly coupled with the proximal ends of both catheters, wherein the flow system is configured to induce a suction flow from the suction catheter and a return flow to the return catheter; and
d. a filter system disposed between the suction catheter and the return catheter and fluidly coupled with the proximal ends of both catheters, wherein the filter system is configured to capture unwanted material extracted from the blood vessel via the suction flow such that it is not returned to the second blood vessel via the return flow.

Enumerated Embodiment 2: The device of enumerated embodiment 1, wherein the flow system comprises a suction pump, a fluidic connection with a vacuum system, and a return pump.

Enumerated Embodiment 3: The device of enumerated embodiment 2, wherein the suction pump and the return pump each comprise roller pumps.

Enumerated Embodiment 4: The device of enumerated embodiment 1, wherein the flow system comprises a manually actuated syringe pump.

Enumerated Embodiment 5: The device of enumerated embodiment 1, wherein the filter system comprises a filter reservoir comprising:
a. a top chamber having a first cone-like structure filter disposed therein, the top chamber comprising an inlet which is fluidly coupled with the suction catheter;
b. a bottom chamber comprising an outlet which is fluidly coupled with the return catheter, and a vacuum inlet fluidly coupled with a vacuum source; and
c. a middle compartment disposed within the bottom chamber, the middle compartment having a second filter disposed therein.

Enumerated Embodiment 6: The device of enumerated embodiment 5, wherein the second filter comprises a funnel-like, cylindrical V-shape structure.

Enumerated Embodiment 7: The device of enumerated embodiment 5, wherein a pore size of the first filter is about 180 μm and a pore size of the second filter is about 40 μm.

Enumerated Embodiment 8: The device of enumerated embodiment 1, wherein the flow rates of the suction flow and the return flow may be independently adjusted.

Enumerated Embodiment 9: The device of enumerated embodiment 1, additionally comprising a console configured to control pressure or flow rates of the suction flow and the return flow based on input from a plurality of sensors.

Enumerated Embodiment 10: The device of enumerated embodiment 9, wherein the console is configured to stop the suction flow, the return flow, or both if a potentially unsafe condition is predicted based on input from the plurality of sensors.

Enumerated Embodiment 11: The device of enumerated embodiment 9, wherein the plurality of sensors comprise flow sensors, bubble sensors, pressure sensors, fluid level sensors, or a combination thereof.

Enumerated Embodiment 12: The device of enumerated embodiment 1, wherein the device is configured to extract the unwanted material without significant overall loss of blood.

Enumerated Embodiment 13: The device of enumerated embodiment 1, wherein the flow system is configured to induce a pulsed flow.

Enumerated Embodiment 14: The device of enumerated embodiment 1, wherein the suction catheter and the return catheter each comprise a through-pathway configured for allowing implements to access through the catheter.

Enumerated Embodiment 15: A suction catheter for extracting unwanted material from a blood vessel, the catheter comprising:
a. a catheter body comprising a proximal end and a distal end, wherein the distal end is configured to be disposed in the blood vessel;
b. a distal opening in the distal end of the catheter body;
c. a suction port and an access port branching from the proximal end of the catheter body, wherein the suction port is configured to be fluidly coupled with a flow system configured to induce a suction flow from the suction catheter, and wherein the access port is configured to allow access through the catheter to the distal opening; and
d. an air-lock coupled with the access port, wherein the air-lock is configured to seal itself or seal around an implement inserted through the access port.

Enumerated Embodiment 16: The catheter of enumerated embodiment 15, wherein a pathway from the distal opening to the suction port has a minimum diameter of at least about 9 mm.

Enumerated Embodiment 17: The catheter of enumerated embodiment 15, wherein the implement comprises a fishing or a daughter catheter configured to be deployed through the catheter body to reach unwanted material from smaller blood vessels.

Enumerated Embodiment 18: The catheter of enumerated embodiment 15, wherein the implement comprises an expandable mesh (154) configured to be deployed through the catheter body to extract unwanted material toward the distal opening.

Enumerated Embodiment 19: A method for removing unwanted material from a blood vessel, the method comprising:
a. providing a device comprising: a suction catheter; a return catheter; a flow system disposed between the suction catheter and the return catheter; and a filter system disposed between the suction catheter and the return catheter;
b. inserting a distal end of the suction catheter into a first blood vessel;
c. inserting a distal end of the return catheter into a second blood vessel;
d. actuating the flow system so as to induce a suction flow from the suction catheter and a return flow to the return catheter; and
e. using the filter system to filter blood removed from the first blood vessel via the suction flow prior to reintroducing it to the second blood vessel via the return catheter, thereby removing the unwanted material.

Enumerated Embodiment 20: The method of enumerated embodiment 19, wherein the first or second blood vessel comprises a left or right femoral vein.

Enumerated Embodiment 21: A device (100) for extracting unwanted material from a blood vessel, the device comprising:
a. a first tube (120) comprising a first end (122) and a second end (124), wherein the first end (122) is configured to be disposed in a first blood vessel and the second end (124) is connected to a reservoir (130);
b. the reservoir (130) comprising: a top chamber (131) having a first filter (132) disposed therein; a bottom chamber (136) comprising an outlet (137), wherein a vacuum inlet (135) connects the bottom chamber middle chamber (133) to a vacuum source, and wherein the bottom chamber (137) is connected to a roller pump; and a middle compartment (133) disposed within the bottom chamber (136), the middle compartment (133) having a second filter (134) and a funnel disposed therein; and
c. a second tube (140) comprising a first end (142) and a second end (144), wherein the first end (144) is connected to the outlet (137) of the reservoir (130) and the second end (142) is configured to be disposed in a second blood vessel.

Enumerated Embodiment 22: The device (100) of enumerated embodiment 21, wherein the top chamber is removable.

Enumerated Embodiment 23: The device (100) of enumerated embodiment 21, wherein the top chamber has multiple access ports.

Enumerated Embodiment 24: The device (100) of enumerated embodiment 21, wherein the second filter (134) comprises a funnel-like structure surrounded by a cylindrical filters.

Enumerated Embodiment 25: The device (100) of enumerated embodiment 21, wherein the vacuum or peristaltic pump controls flow of blood out of a patient.

Enumerated Embodiment 26: The device (100) of enumerated embodiment 21, wherein the roller pump controls flow of blood into a patient.

Enumerated Embodiment 27: The device (100) of enumerated embodiment 21, wherein the device (100) further comprises a console (200) to control pressure or flow rate of the device, and level sensors to control the volume of blood in the reservoir (130).

Enumerated Embodiment 28: The device (100) of enumerated embodiment 27, wherein the console (200) further comprises a pedal (210) to control the pressure or flow rate of the device.

Enumerated Embodiment 29: The device (100) of enumerated embodiment 27, wherein the pressure is between about 0 mmHg to −700 mmHg.

Enumerated Embodiment 30: The device (100) of enumerated embodiment 21, wherein the first blood vessel is a right or left femoral vein.

Enumerated Embodiment 31: The device (100) of enumerated embodiment 21, wherein the second blood vessel is a right or left femoral vein.

Enumerated Embodiment 32: The device (100) of enumerated embodiment 21, wherein the unwanted material is a foreign body, a clot, or other infectious material.

Enumerated Embodiment 33: The device (100) of enumerated embodiment 21, wherein the first tube (120) has a length between about 140 cm to 170 cm.

Enumerated Embodiment 34: The device (100) of enumerated embodiment 21, wherein a shape of the second filter (134) is configured to create laminar flow of blood from the reservoir (130) to the second tube (140).

Enumerated Embodiment 35: The device (100) of enumerated embodiment 34, wherein the second filter (134) is conical or funnel-like in shape.

Enumerated Embodiment 36: The device (100) of enumerated embodiment 21, wherein a pore size of the first filter (132) is about 180 μm.

Enumerated Embodiment 37: The device (100) of enumerated embodiment 21, wherein a pore size of the second filter (134) is about 40 μm.

Enumerated Embodiment 38: The device (100) of enumerated embodiment 21, wherein the top chamber (131) is transparent and is configured to be opened during or post operation of the device (100).

Enumerated Embodiment 39: The device (100) of enumerated embodiment 21, wherein the top chamber (131) is accessible to visualize and to collect the filtered unwanted material.

Enumerated Embodiment 40: The device (100) of enumerated embodiment 21, wherein the device further comprises a fishing or a daughter catheter that is deployed from inside the first tube (120) to reach unwanted materials in smaller vessels.

Enumerated Embodiment 41: The device (100) of enumerated embodiment 21, wherein the device further comprises a screw-in lock cap disposed at the first end of the suction catheter (120) and at the end of the return catheter.

Enumerated Embodiment 42: The device (100) of enumerated embodiment 41, wherein a fishing catheter (150) or a daughter catheter is configured to be deployed through the screw-in lock cap to extract the unwanted material in smaller vessels.

Enumerated Embodiment 43: The device (100) of enumerated embodiment 42, wherein the mesh (154) is configured to be expanded when needed and in multiple configurations as desired by a user.

Enumerated Embodiment 44: A device (100) for extracting an embolism from a blood vessel, the device comprising:
a. a first catheter (120) comprising a first end (122) and a second end (124), wherein the first end (122) is configured to be disposed in a first blood vessel and the second end (124) is connected to a reservoir (130);
b. the reservoir (130) having a filter disposed therein, wherein the reservoir (130) is connected to a vacuum pump and a roller pump, wherein an outlet (137) is disposed at a bottom end of the reservoir (130); and
c. a second catheter (140) comprising a first end (144) and a second end (142), wherein the first end (144) is connected to the outlet (137) of the reservoir (130) and the second end (142) is configured to be disposed in a second blood vessel.

Enumerated Embodiment 45: The device of enumerated embodiment 44, wherein the device (100) further comprises a console (200).

Enumerated Embodiment 46: The device of enumerated embodiment 45 wherein the console (200) regulates a pressure of the vacuum pump, and engagement of the roller pump.

Enumerated Embodiment 47: The device of enumerated embodiment 45, wherein the console (200) regulates a flowrate of blood being suctioned from a patient.

Enumerated Embodiment 48: The device of enumerated embodiment 45, wherein the console (200) regulates a flowrate of blood being returned to a patient.

Enumerated Embodiment 49: The device (100) of enumerated embodiment 45, wherein the console (200) further comprises a pedal (210) to control the pressure or flowrate of the device.

Enumerated Embodiment 50: The device (100) of enumerated embodiment 46, wherein the pressure is between about 0 mmHg to −700 mmHg.

Enumerated Embodiment 51: The device (100) of enumerated embodiment 44, wherein blood is aspirated from a patient in a volume up to about 1.5 L.

Enumerated Embodiment 52: The device (100) of enumerated embodiment 44, wherein a flowrate of the blood being suctioned from a patient is between about 200 mL/min to 3 L/min.

Enumerated Embodiment 53: The device (100) of enumerated embodiment 44, wherein a flowrate of the blood being returned to a patient is between about 200 mL/min to 3 L/min.

Enumerated Embodiment 54: The device (100) of enumerated embodiment 44, wherein a flow rate of blood being suctioned from a patient is different from a flow rate of blood being returned to the patient.

Enumerated Embodiment 55: The device (100) of enumerated embodiment 44, wherein the reservoir (130) further comprises a second inlet disposed at a top of the reservoir (130).

Enumerated Embodiment 56: The device (100) of enumerated embodiment 44, wherein a therapeutic composition, saline, or additional blood products is administered to available ports connected to the device (100).

Enumerated Embodiment 57: The device (100) of enumerated embodiment 44, wherein the device further comprises a fishing or a daughter catheter that is deployed from inside the first catheter (120) to extract unwanted materials in smaller vessels.

Enumerated Embodiment 58: The device (100) of enumerated embodiment 44, wherein the device further comprises a screw-in lock cap disposed at the first end of the first catheter (120).

Enumerated Embodiment 59: The device (100) of enumerated embodiment 58, wherein a fishing cather (150) or a daughter catheter is configured to be deployed through the screw-in lock cap to extract the unwanted material in smaller vessels.

Enumerated Embodiment 60: The device (100) of enumerated embodiment 58, wherein the mesh (154) is configured to be expanded when needed and in multiple configurations as desired by a user.

Enumerated Embodiment 61: A method for removing unwanted material from a blood vessel using the device (100) of enumerated embodiment 21, the method comprising:
a. connecting the first end (122) of the first tube (120) to the first blood vessel;
b. connecting the second end (142) of the second tube (140) to the second blood vessel;
c. connecting the device (100) to a console (200), a vacuum pump, and two roller pumps;
d. using the console (200) to apply a first pressure on the first tube (120) with the vacuum pump, thereby suctioning unwanted materials and blood from the heart or blood vessel to the first tube (120);
e. filtering the blood through the reservoir (130), wherein the unwanted material is filtered from the blood;
f. using the console (200) to apply a second pressure at the bottom of the reservoir (130) using the roller pump, facilitating laminar flow of blood from the reservoir to the second tube (140); and
g. returning the blood to the patient through the second tube (140).

Enumerated Embodiment 62: The method of enumerated embodiment 61, wherein the console (200) is configured to execute computer-readable instructions for controlling the first pressure and second pressure.

Enumerated Embodiment 63: The method of enumerated embodiment 61, wherein the console (200) is configured to execute computer-readable instructions for controlling a flow rate of the device.

Enumerated Embodiment 64: The method of enumerated embodiment 61, wherein the console (200) uses patient data to build safety measures of the device.

Enumerated Embodiment 65: The method of enumerated embodiment 62, wherein the console will stop the drainage when a patient MAP drops below 65 mmHg or greater than 10 mmHg of baseline.

Enumerated Embodiment 66: A method for removing unwanted material from a blood vessel using the device (100) of enumerated embodiment 44, the method comprising:
a. connecting the first end (122) of the catheter (120) to the first blood vessel;
b. connecting the second end (142) of the second catheter (140) to the second blood vessel;
c. connecting the device (100) to a console (200);
d. using the console (200) to apply a first pressure on the first catheter (120) with the vacuum pump, thereby suctioning blood from the blood vessel to the first catheter (120);
e. filtering the blood through the reservoir (130), wherein the unwanted material is filtered from the blood;
f. using the console (200) to apply a second pressure at the bottom of the reservoir (130) using the roller pump, facilitating laminar flow of blood from the reservoir to the second catheter (140); and
g. returning the blood to the patient through the second catheter (140).

Enumerated Embodiment 67: The method of enumerated embodiment 66, wherein the console (200) is configured to execute computer-readable instructions for controlling the first pressure and second pressure.

Enumerated Embodiment 68: The method of enumerated embodiment 66, wherein the console (200) is configured to execute computer-readable instructions for controlling a flow rate of the device.

Enumerated Embodiment 69: The method of enumerated embodiment 66, wherein the console (200) uses patient data to build safety measures of the device.

Enumerated Embodiment 70: The method of enumerated embodiment 66, wherein a fishing or a daughter catheter can be deployed inside the first tube (120) to reach foreign materials in the smaller vessels.

Enumerated Embodiment 71: The method of enumerated embodiment 66, wherein a screw-in lock cap is disposed at the first end of the first catheter (120).

Enumerated Embodiment 72: The method of enumerated embodiment 71, wherein a fishing catheter is configured to be deployed through the screw-in lock cap to extract unwanted material toward the first tube (120).

Enumerated Embodiment 73: The method of enumerated embodiment 71, wherein the mesh of the fishing catheter is configured to be expanded when needed and in multiple configurations as desired by a user.

Enumerated Embodiment 74: A method for removing unwanted material from a heart or blood vessel, the method comprising:
a. connecting a first end (122) of a first catheter (120) to a first blood vessel and a second end (124) of the first catheter to a reservoir (130);
b. connecting a second end (142) of a second catheter (140) to a second blood vessel and a first end (144) of the second catheter to the reservoir (130);
c. applying a first pressure on the first catheter (120) via a first pump, thereby suctioning unwanted materials and blood from the heart or blood vessel to the first catheter (120);
d. filtering the blood through the reservoir (130), wherein the unwanted material is filtered from the blood;
e. applying a second pressure at the bottom of the reservoir (130) using a second pump, thereby facilitating laminar flow of blood from the reservoir to the second catheter (140); and
f. returning the blood to the patient through the second catheter (140).

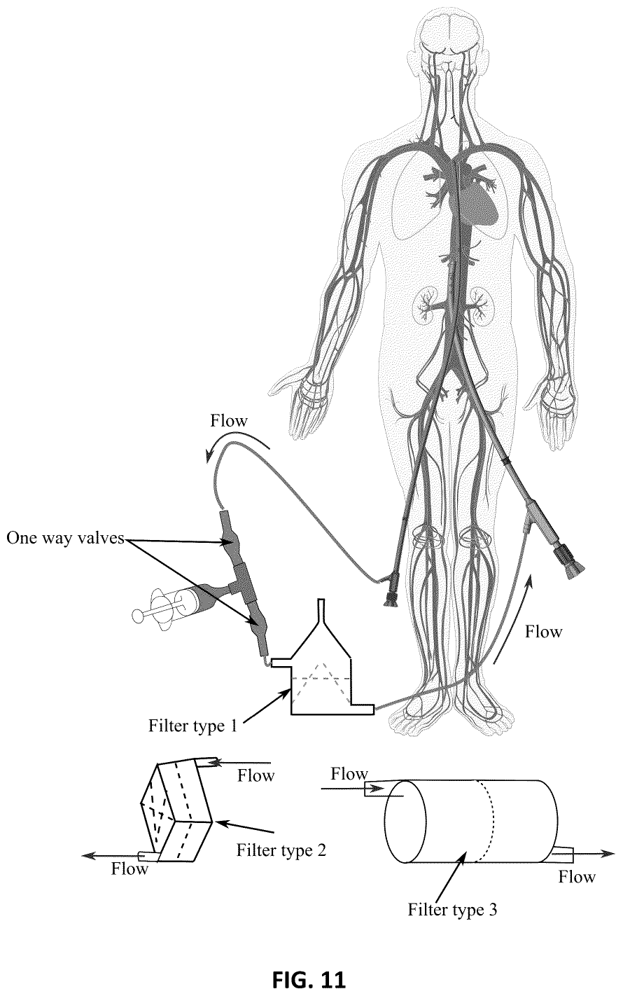

Enumerated Embodiment 74: A method for manually removing unwanted material from a heart or blood vessel, the method comprising:
a. fluidly connecting a first end (122) of a first catheter (120) to a first blood vessel and a second end (124) of the first catheter to a manual syringe pump consisting of two one-way valves (FIG. 11);
b. The lockable syringe with quick-connect to the T-junction can be used to prime the apparatus either with patient blood or available crystalloid.
c. connecting a the manual syringe pump to the primed filter reservoir;
d. the prime reservoir is connected to the second catheter (140).
e. When the syringe is pulled and lock, it will generate negative suction to extract unwanted materials and blood through the first one way valves.
f. The syringe is then pushed to transfer unwanted materials and blood through the second one-way valve to the filtering reservoir, wherein the unwanted material is filtered from the blood;
a. the displacement of volume by the syringe also facilitates blood flow from the reservoir to the second catheter (140); and returning the blood to the patient through the second catheter (140).

The reference numbers recited in the below claims are solely for ease of examination of this patent application, and are exemplary, and are not intended in any way to limit the scope of the claims to the particular features having the corresponding reference numbers in the drawings.

What is claimed is:

1) A device for extracting unwanted material comprising clots in a pulmonary artery, the device comprising:
a) a suction catheter comprising a proximal end and a distal end, wherein the distal end is configured to be disposed in a first blood vessel;
b) a return catheter comprising a proximal end and a distal end, wherein the distal end is configured to be disposed in a second blood vessel;
c) a flow system disposed between the suction catheter and the return catheter and fluidly coupled with the proximal ends of both catheters, wherein the flow system is configured to induce a suction flow from the suction catheter and a return flow to the return catheter;
d) a two stage filter system disposed between the suction catheter and the return catheter and fluidly coupled with the proximal ends of both catheters, comprising:
i) a first stage filter comprising a conical pyramid structure configured to enhance visibility of the clots;
ii) a second stage filter comprising a housing, a solid funnel structure disposed within the housing, and a half-walled sliding spill plate disposed below the solid funnel structure; and
iii) a slope structure disposed below the second stage filter;
wherein the filter system is configured to capture unwanted material in blood extracted from the first blood vessel via the suction flow such that the unwanted material is captured on the first stage filter and blood is further filtered in the second stage;
wherein the spill plate comprises a 45 degree sloped shape on a first half and a wall having a height of 1 cm on a second half opposite the first half such that laminar flow is achieved in the blood in one direction as the blood passes through the solid funnel structure into the spill plate and onto the slope;
wherein only filtered and de-aired blood is returned via the return catheter;
e) a plurality of sensors operatively coupled to the filter system, configured to measure negative pressure in the filter system, a flow rate of the blood into the filter system, and a fluid level of the blood in the filter system; and
f) a console operatively coupled to the filter system and the plurality of sensors, configured with computer readable instructions and algorithms to control pressure or flow rates of the suction flow and the return flow based on the fluid level of the blood in the filter system such that the suction flow, the return flow, or a combination thereof are stopped by the console when bubbles are detected, when the fluid level is below a threshold, or a combination thereof.

2) The device of claim 1, wherein the flow system comprises a suction pump, a fluidic connection with a vacuum system, and a return pump.

3) The device of claim 2, wherein the suction pump and the return pump each comprise roller pumps.

4) The device of claim 1, wherein the suction and return flow system comprises a manually actuated 100 ml syringe pump with 2 one way valves before and after the syringe pump, wherein the syringe pump comprises a large bore luer lock port, wherein the syringe pump is configured to lock when fully pulled to generate negative pressure, wherein the syringe pump is configured to act as a displacement pump and move clots and blood from the suction catheter to the filter system such that de-aired and filtered blood is returned through the console, another manual syringe apparatus, or a combination thereof.

5) The device of claim 1, wherein the filter system further comprises a filter reservoir comprising:
a) a top chamber comprising a first conical pyramid filter disposed therein and an inlet fluidly coupled with the suction catheter;
b) a bottom chamber comprising an outlet fluidly coupled with the return catheter and a vacuum inlet fluidly coupled with a vacuum source;
c) a middle compartment disposed within the bottom chamber, the middle compartment comprising a second filter disposed therein; and
d) a hardshell reservoir configured to withstand high negative pressure, comprising a pressure release valve configured to release at −760 mmHg or less;
wherein the top chamber is removable to access unwanted materials.

6) The device of claim 5, wherein the second filter comprises a cylindrical funnel structure connecting to the first filter to generate laminar blood flow toward the spill plate to de-air blood.

7) The device of claim 5, wherein a pore size of the first filter is about 180 μm and a pore size of the second filter is about 40 μm.

8) The device of claim 1, wherein flow rates of the suction flow and the return flow are configured to be independently adjusted.

9) The device of claim 1, wherein the device is configured to extract the unwanted material without significant overall loss of blood.

10) The device of claim 1, wherein the device is configured to rapidly infuse a volume of blood.

11) The device of claim 1, wherein the suction catheter and the return catheter each comprise a through-pathway configured for allowing implements to access through the catheter.

12) A suction catheter for extracting unwanted material from blood extracted from a blood vessel, the catheter comprising:
a) a catheter body comprising a proximal end and a distal end, wherein the distal end is configured to be disposed in the blood vessel, wherein the proximal end is configured to be fluidly coupled to a filter system comprising a solid funnel structure disposed within and a half-walled sliding spill plate disposed below the solid funnel structure, wherein the spill plate has a sloped shape on a first end and a wall at a second end opposite the first end such that laminar flow is achieved in the blood in one direction as the blood passes through the solid funnel structure into the spill plate and onto a slope disposed at a bottom of the filter system;
b) a distal opening in the distal end of the catheter body comprising a screw cap end containing a flexible rubberized insert configured to compress inserted objects;
c) a side suction port and an access port branching from the proximal end of the catheter body, wherein the side suction port is configured to be fluidly coupled with a flow system configured to induce a suction flow of the blood from the suction catheter, and wherein the access port is configured to allow access through the catheter to the distal opening; and
d) an air-lock coupled with the access port, wherein the air-lock is configured to seal itself or seal around an implement inserted through the access port;
wherein the catheter operatively coupled to the filter system is configured to be controlled by a plurality of sensors configured to monitor negative vacuum pressure in the filter system, a flow rate of the blood into the filter system, and a fluid level of the blood in the filter system, and a console operatively coupled to the filter system and the plurality of sensors, configured to execute computer readable instructions for controlling pressure or flow rate of the suction flow in the filter system such that the suction flow is stopped by the console when bubbles are detected or when the fluid level is above a maximum threshold, increasing the suction flow when the fluid level is below a minimum threshold, or a combination thereof.

13) The catheter of claim 12, wherein a pathway from the distal opening to the suction port has a minimum diameter of at least about 9 mm.

14) The catheter of claim 12, wherein the implement comprises a tapered daughter catheter configured to be deployed through the catheter body to reach unwanted material from smaller blood vessels, the tapered daughter catheter comprising:
a) a dilator with recessed canal to fit a metal rod;
wherein the dilator comprises a length of about 160 cm and comprises a smooth blunted end;
b) the metal rod comprising a length of about 126 cm; and
c) a ball connector disposed at a distal tip of the tapered daughter catheter;
wherein the tapered daughter catheter is about 40 cm long such that the tapered daughter catheter fits to the suction catheter;
wherein the tapered daughter catheter size tapers down to a diameter of 10 Fr or more.

15. A fishing catheter configured to be deployed through a delivery catheter disposed in a vessel, configured to extract unwanted material toward the suction catheter, the fishing catheter comprising:
a) an expandable mesh comprising 8 moldable resilient metal nitinol wires, wherein each wire is about 1 mm in thickness, 1 mm in width, and 30 mm in length, wherein the expandable mesh is configured to shift into an open configuration upon actuation, wherein a diameter of the mesh in the open configuration is 18 mm;
b) a dilator comprising a distal end coupled to the 8 wires;
c) a sliding catheter comprising a proximal end coupled to the 8 wires;
wherein the 8 wires are coupled to the dilator, the sliding catheter, or a combination thereof at a 30-degree angle such that the mesh rotates to form a helical circular disk;
d) a sheath component coupled to the mesh, configured to cover the mesh as the mesh is retracted during insertion into the vessel, wherein the sheath is de-aired upon insertion into the vessel;
wherein the mesh is configured to deploy by a deployment process comprising:
i) pulling the sheath to uncover the mesh; and
ii) pushing the delivery catheter forward towards the distal end of the dilator in a circular fashion;
wherein the mesh comprises a satellite-shape mesh structure, an umbrella-shape mesh structure, or a helical-shape structure.

16) A method for removing unwanted material from a left or right femoral blood vessel, the method comprising:
a) providing a device comprising:
i) a suction catheter;
ii) a return catheter;
iii) a flow system disposed between the suction catheter and the return catheter;
iv) a filter system disposed between the suction catheter and the return catheter, the filter system comprising a solid funnel structure disposed within and a spill plate disposed below the filter system, wherein the spill plate has a sloped shape on a first end and a wall at a second end opposite the first end such that laminar flow is achieved in the blood in one direction as the blood passes through the solid funnel structure into the spill plate and onto a slope disposed at a bottom of the filter system;
v) a plurality of sensors operatively coupled to the filter system; and
vi) a console operatively coupled to the filter system and the plurality of sensors;
b) inserting a distal end of the suction catheter into a first blood vessel;
c) inserting a distal end of the return catheter into a second blood vessel;
d) actuating the flow system so as to induce a suction flow from the suction catheter and a return flow to the return catheter;
e) using the filter system to filter the blood removed from the first blood vessel via the suction flow prior to reintroducing it to the second blood vessel via the return catheter, thereby removing the unwanted material;
f) measuring, by the plurality of sensors, negative vacuum pressure in the filter system, a flow rate of the blood into the filter system, and a fluid level of the blood in the filter system; and
g) stopping, by the console, the suction flow and the return flow when bubbles are detected, when the fluid level is below a threshold, or a combination thereof.

17) A device for extracting unwanted material from a blood vessel, the device comprising:
a) a suction catheter comprising a proximal end and a distal end, wherein the distal end is configured to be disposed in a first blood vessel;
b) a return catheter comprising a proximal end and a distal end, wherein the distal end is configured to be disposed in a second blood vessel;
c) a flow system disposed between the suction catheter and the return catheter and fluidly coupled with the proximal ends of both catheters, wherein the flow system is configured to induce a suction flow from the suction catheter and a return flow to the return catheter;
d) a filter system disposed between the suction catheter and the return catheter and fluidly coupled with the proximal ends of both catheters, the filter system comprising a solid funnel structure disposed within, and a spill plate disposed below the solid funnel structure, wherein the filter system is configured to capture unwanted material in blood extracted from the blood vessel via the suction flow such that the unwanted material is not returned to the second blood vessel via the return flow, wherein the spill plate has a sloped shape on a first end and a wall at a second end opposite the first end such that laminar flow is achieved in the blood in one direction as the blood passes through the solid funnel structure into the spill plate and onto a slope disposed at a bottom of the filter system;
e) a plurality of sensors operatively coupled to the filter system, configured to measure negative pressure in the filter system, a flow rate of the suction flow and a flow rate of the return flow, and a fluid level of the blood in the filter system; and
f) a console operatively coupled to the filter system and the plurality of sensors, comprising a processor configured to execute computer readable instructions and a memory component operatively coupled to the processor, comprising computer-readable instructions for:
i) intaking, by the plurality of sensors, data on the suction flow, the return flow, reservoir weight, bubble presence, level sensors comprising a lowest level sensor, a middle level sensor, and a highest level sensor, and patient parameters;
ii) adjusting thresholds of the data in real-time tailoring to a condition or sequence of the procedure;
iii) detecting, by the plurality of sensors, whether or not bubbles are present in the filter system and whether or not the lowest level sensor is appropriate to engage;
iv) stopping, if the bubbles are detected or the lowest level sensor is appropriate to engage, the suction flow and the return flow;
v) determining, by the plurality of sensors, whether the flow rate of the suction flow is equal to the flow rate of the return flow;
vi) adjusting, if the flow rate of the suction flow is not equal to the flow rate of the return flow, the flow rate of the suction flow and the flow rate of the return flow such that the flow rate of the suction flow is equal to the return flow;
vii) simultaneously adjusting, if the flow rate of the suction flow is not equal to the flow rate of the return flow, the return flow and a vacuum for suction flow;
viii) determining, by the plurality of sensors, the fluid level within the filter system;
ix) stopping, if the fluid level within the filter system is below a fluid level threshold, the suction flow and the return flow;
x) intaking and assessing, if patient parameters require more volume; and
xi) adjusting the suction flow and return flow to restore patient hemodynamics.
